(19) United States
(12) Patent Application Publication
(10) Pub. No.: US 2014/0051146 A1
(43) Pub. Date: Feb. 20, 2014

(54) METHOD

(71) Applicant: Susan Mampusti Madrid, Millbrae, CA (US)

(72) Inventor: Susan Mampusti Madrid, Millbrae, CA (US)

(73) Assignee: DUPONT NUTRITION BIOSCIENCES APS, Copenhagen K (DK)

(21) Appl. No.: 14/000,933

(22) PCT Filed: Feb. 16, 2012
(86) PCT No.: PCT/IB2012/050711

§ 371 Date: Aug. 22, 2013

Related U.S. Application Data
(60) Provisional application No. 61/445,657, filed on Feb. 23, 2011.

(51) Int. Cl. C12N 9/18

(52) U.S. Cl. CPC C12N 9/18
USPC 435/197; 435/254.11; 435/254.2; 435/254.21; 435/254.22; 435/254.23; 435/254.3; 435/254.4; 435/254.5; 435/254.6; 435/254.7; 435/254.8; 435/320.1

(57) ABSTRACT

In one aspect there is provided a method for producing a recombinant enzyme capable of hydrolysing chlorophyll or a chlorophyll derivative, comprising a step of intracellular expression of the recombinant enzyme in a eukaryotic host cell.

### FIELD

The present invention relates to the production of enzymes capable of hydrolysing chlorophyll and chlorophyll derivatives.

### BACKGROUND

Chlorophyll is a green-coloured pigment widely found throughout the plant kingdom, in algae and cyanobacteria. Chlorophyll is essential for photosynthesis and is one of the most abundant organic metal compounds found on earth. Thus many products derived from plants, including foods and feeds, contain significant amounts of chlorophyll.

For example, vegetable oils derived from oilseeds such as soybean, palm or rape seed (canola), cotton seed, sunflower seed, grape seed and peanut typically contain some chlorophyll. However the presence of high levels of chlorophyll pigments in vegetable oils is generally undesirable. This is because chlorophyll imparts an undesirable green colour and can induce oxidation of oil during storage, leading to a deterioration of the oil.

Various methods have been employed in order to remove chlorophyll from vegetable oils. Chlorophyll may be removed during many stages of the oil production process, including the seed crushing, oil extraction, degumming, caustic treatment and bleaching steps. However the bleaching step is usually the most significant for reducing chlorophyll residues to an acceptable level. During bleaching the oil is heated and passed through an adsorbent to remove chlorophyll and other colour-bearing compounds that impact the appearance and/or stability of the finished oil. The adsorbent used in the bleaching step is typically clay.

In the edible oil processing industry, the use of such steps typically reduces chlorophyll levels in processed oil to between 0.02 to 0.05 ppm. However the bleaching step increases processing cost and reduces oil yield due to entrainment in the bleaching clay. The use of clay may remove many desirable compounds such as carotenoids and tocopherol from the oil. Also the use of clay is expensive, this is particularly due to the treatment of the used clay (i.e. the waste) which can be difficult, dangerous (prone to self-ignition) and thus costly to handle. Thus attempts have been made to remove chlorophyll from oil by other means, for instance using the enzyme chlorophyllase.

In plants, chlorophyllase (chlase) is thought to be involved in chlorophyll degradation and catalyzes the hydrolysis of an ester bond in chlorophyll to yield chlorophyllide and phytol. WO 2006009676 describes an industrial process in which chlorophyll contamination can be reduced in a composition such as a plant oil by treatment with chlorophyllase. The water-soluble chlorophyllide which is produced in this process is also green in colour but can be removed by an aqueous extraction or silica treatment.

Chlorophyll is often partly degraded in the seeds used for oil production as well as during extraction of the oil from the seeds. One common modification is the loss of the magnesium ion from the porphyrin (chlorin) ring to form the derivative known as pheophytin (see FIG. 1). The loss of the highly polar magnesium ion from the porphyrin ring results in significantly different physico-chemical properties of pheophytin compared to chlorophyll. Typically pheophytin is more abundant in the oil during processing than chlorophyll. Pheophytin has a greenish colour and may be removed from the oil by an analogous process to that used for chlorophyll, for instance as described in WO 2006009676 by an esterase reaction catalyzed by an enzyme having a pheophytinase activity. Under certain conditions, some chlorophyllases are capable of hydrolyzing pheophytin as well as chlorophyll, and so are suitable for removing both of these contaminants. The products of pheophytin hydrolysis are the red/brown-colored pheophorbide and phytol. Pheophorbide can also be produced by the loss of a magnesium ion from chlorophyllide, i.e. following hydrolysis of chlorophyll (see FIG. 1). WO 2006009676 teaches removal of pheophorbide by an analogous method to chlorophyllide, e.g. by aqueous extraction or silica adsorption.

Pheophytin may be further degraded to pyropheophytin, both by the activity of plant enzymes during harvest and storage of oil seeds or by processing conditions (e.g. heat) during oil refining (see “Behaviour of Chlorophyll Derivatives in Canola Oil Processing”, JAOCS, Vol, no. 9 (September 1993) pages 837-841). One possible mechanism is the enzymatic hydrolysis of the methyl ester bond of the isocyclic ring of pheophytin followed by the non-enzymatic conversion of the unstable intermediate to pyropheophytin. A 28-29 kDa enzyme from Chenopodium album named pheophorbidase is reportedly capable of catalyzing an analogous reaction on pheophorbide, to produce the phytol-free derivative of pyropheophytin known as pyropheophorbide (see FIG. 1). Pyropheophorbide is less polar than pheophorbide resulting in the pyropheophoribe having a decreased water solubility and an increased oil solubility compared with pheophorbide.

Depending on the processing conditions, pyropheophytin can be more abundant than both pheophytin and chlorophyll in vegetable oils during processing (see Table 9 in volume 2.2. of Bailey's Industrial Oil and Fat Products (2005), 6^{th} edition, Ed. by Fereidoon Shahidi, John Wiley and Sons). This is partly because of the loss of magnesium from chlorophyll during harvest and storage of the plant material. If an extended heat treatment at 90° C. or above is used, the amount of pyropheophytin in the oil is likely to increase and could be higher than the amount of pheophytin. Chlorophyll levels are also reduced by heating of oil seeds before pressing and extraction as well as the oil degumming and alkali treatment during the refining process. It has also been observed that phospholipids in the oil can complex with magnesium and thus reduce the amount of chlorophyll. Thus chlorophyll is a less abundant contaminant compared to pyropheophytin (and pheophytin) in many plant oils.

There is therefore a need for methods for large-scale production of chlorophyllases and related enzymes, particularly for use in oil refining. A chlorophyllase from Triticum aestivum (wheat) has been expressed recombinantly in Escherichia coli, as described in Arkus et al. (2005), Arch. Biochem. Biophys 438:146-155. However, the yield of recombinant chlorophyllase obtained by such bacterial expression methods is low, typically in the order of a few milligrams per liter.

There is a still a need for an improved method for the production of chlorophyllases and related enzymes, in particular for rapid large-scale expression of enzymes suitable for use in refining of plant oils.

### SUMMARY

In one aspect, the present invention provides a method for producing, preferably in high yield, a recombinant enzyme capable of hydrolysing chlorophyll or one or more chlorophyll derivatives, comprising a step of intracellular expression of the recombinant enzyme in a host cell, e.g. a microbial and/or eukaryotic host cell. The host cell is typically a eukaryotic organism, including yeasts such as Saccharomyces sp., Pichia sp, Hansenula and filamentous fungi such as Aspergillus sp., Fusarium sp., and Chrysosporium.

In one embodiment, the host cell is a fungal cell such as Trichoderma, Aspergillus sp, Pichia sp, Hansenula. Saccharomyces sp., Fusarium sp. Preferably the host cell is derived from Trichoderma sp, e.g. Trichoderma reesei.

In specific embodiments, the recombinant enzyme has chlorophyllase, pheophytinase and/or pyropheophytinase activity, e.g. chlorophyllase activity.

The recombinant enzyme may comprise one, two or three amino acid sequences selected from GHSXGG (SEQ ID NO:36), DPVXG (SEQ ID NO:37) and YGHXD (SEQ ID NO:38).

In some embodiments, the gene encoding the recombinant enzyme is derived from a plant, such as Arabidopsis thaliana, Brassica oleracea, Ricinus communis, Ginkgo biloba, Populus trichocarpa, Vitis vinifera, Phyllostachys heterocycla, Sorghum bicolor, Glycine max, Pachira macrocarpa, Triticum aestivum, Citrus sinensis, Zea mays, Chenopodium album, Picea sitchensis or algae, such as Chlamydomonas reinhardtii.

Preferably the recombinant enzyme comprises a polypeptide sequence as defined in any one of SEQ ID NO:s 1 to 18, or a functional fragment or variant thereof. By “functional fragment or variant” it is meant a fragment or variant which is a functional enzyme, e.g an enzyme having chlorophyllase, pheophytinase and/or pyropheophytinase activity. Typically a functional fragment or variant has at least 75% sequence identity to any one of SEQ ID NO:s 1 to 18 over at least 50 amino acid residues.

Preferably the recombinant enzyme is encoded by a nucleic acid sequence as defined in any one of SEQ ID NO:s 19 to 35, or a functional fragment or variant thereof. By “functional fragment or variant” it is typically meant a fragment or variant which encodes a functional enzyme, e.g an enzyme having chlorophyllase, pheophytinase and/or pyropheophytinase activity. Typically functional fragments or variants have at least 75% sequence identity to any of SEQ ID NO:s 19 to 35 over at least 50 nucleotide residues.

In one embodiment, the method further comprises lysing the host cells and recovering the recombinant enzyme from the lysate. Preferably the host cells are lysed using a detergent. Preferably the detergent treatment is combined with a heat treatment to selectively recover the recombinant enzyme. In an alternative embodiment, the host cells are treated with an organic acid, for instance in order to kill the cells. Preferably the organic acid treatment is combined with a heat treatment step. In another embodiment, the host cells are lysed by homogenization, optionally in combination with a detergent, organic acid or heat treatment step.

In a further aspect, the invention provides an expression vector, comprising a nucleic acid sequence encoding an enzyme capable of hydrolysing chlorophyll or a chlorophyll derivative, operably linked to one or more control sequences suitable for directing intracellular expression of the enzyme in a host cell, e.g. a microbial and/or eukaryotic host cell. Preferably the expression vector is suitable for expressing the enzyme in a eukaryotic host cell.

Preferably the expression vector comprises a nucleic acid sequence as defined in any one of SEQ ID NO:s 19 to 34, or a functional fragment or variant thereof having at least 75% sequence identity to any of SEQ ID NO:s 19 to 35 over at least 50 nucleotide residues.

In a further aspect, the present invention provides a (e.g. microbial and/or eukaryotic) host cell comprising an expression vector as defined above. Preferably the host cell is a fungal cell.

In a further aspect, the present invention provides a recombinant enzyme capable of hydrolyzing chlorophyll or one or more chlorophyll derivatives, which is obtainable by a method as defined above, e.g. by introducing an expression vector containing the chlorophyllase gene of interest as defined above into a (e.g. microbial and/or eukaryotic) host cell.

Preferably the recombinant enzyme comprises a polypeptide sequence as defined in any one of SEQ ID NO:s 1 to 18, or a functional fragment or variant thereof having at least 75% sequence identity to any one of SEQ ID NO:s 1 to 18 over at least 50 amino acid residues.

### BRIEF DESCRIPTION OF DRAWINGS

FIG. 1 shows the reactions involving chlorophyll and derivatives and enzymes produced in the present invention.

FIG. 2A shows a dendrogram based on the protein sequence similarity to AtCLH2 and other known and putative chlorophyllases. Chlorophyllases from monocots (grasses such as wheat, bamboo, sorghum, zea mays) are clustered together while the 2 Brassica oleracea chlorophyllases are in two separate clusters.

FIG. 2B shows an alignment of amino acid sequences from various chlorophylases. The conserved catalytic residues (Ser-His-Asp) are indicated by . Conserved motifs around the active site residues, aspartate and histidine are unique to the chlorophyllases. The first motif GHSXGG containing the active site serine is common to other esterases such as lipases.

FIG. 3 shows an expression construct for production of wheat chlorophyllase in fungal cells (Trichoderma). The strong Cbhl promoter is used to drive the expression of chlorophyllase with and without the different signal peptides.

FIG. 4 shows screening of fungal strains (1-12) for expression of Triticum aestivum chlorophyllase. SDS-PAGE of intracellular cell extracts from different transformants showing different expression levels of a 34 kDa recombinant wheat chlorophyllase (arrows). C=untransformed strain as control.

FIG. 5 shows extracellular accumulation of recombinant wheat chlorophyllase (CORE). SDS-PAGE showing increased extracellular deposition of chlorophyllase with fermentation time.

FIG. 6 shows intracellular accumulation of recombinant wheat chlorophyllase showing peak of protein production at 69 hours.

FIG. 7 shows an expression construct for the production of bamboo chlorophyllase, and SDS-PAGE showing different transformants producing the recombinant protein. Strains 3, 5, 6, 11, 12, 13, 14, 15, 16, 17, and 18 showed a band cross reacting with the antibody towards wheat chlorophyllase.

FIG. 8 shows expression constructs used to transform fungal cells. These constructs contain synthetic genes encoding chlorophyllases from Brassica, castor bean and Glycine max.

FIG. 9 shows screening of strains expressing chlorophyllases from Brassica (1-6), Castor bean (7-14) and Glycine max (15-23). Different transformants showed varying levels of the recombinant protein. An antibody towards wheat chlorophyllase was used to identify the chlorophyllases from other plants.

FIG. 10 shows constructs containing the synthetic genes encoding chlorophyllases from Pachira and Poplar. Transformants #24-32 showed low expression level for the Pachira chlorophyllase compared with higher levels of expression for Poplar chlorophyllase expressing strains 33-40.

FIG. 11 shows strains 41-49 with detectable expression levels for the chlorophyllase gene from Vitis vinifera.

FIGS. 12 to 29 show chlorophyllase amino acid sequences for expression in Trichoderma reesei. The underlined amino acid sequences were deleted for the production of truncated versions of the protein.

FIGS. 30 to 46 show synthetic gene sequences encoding chlorophyllases with codons optimized for expression in fungal production hosts.

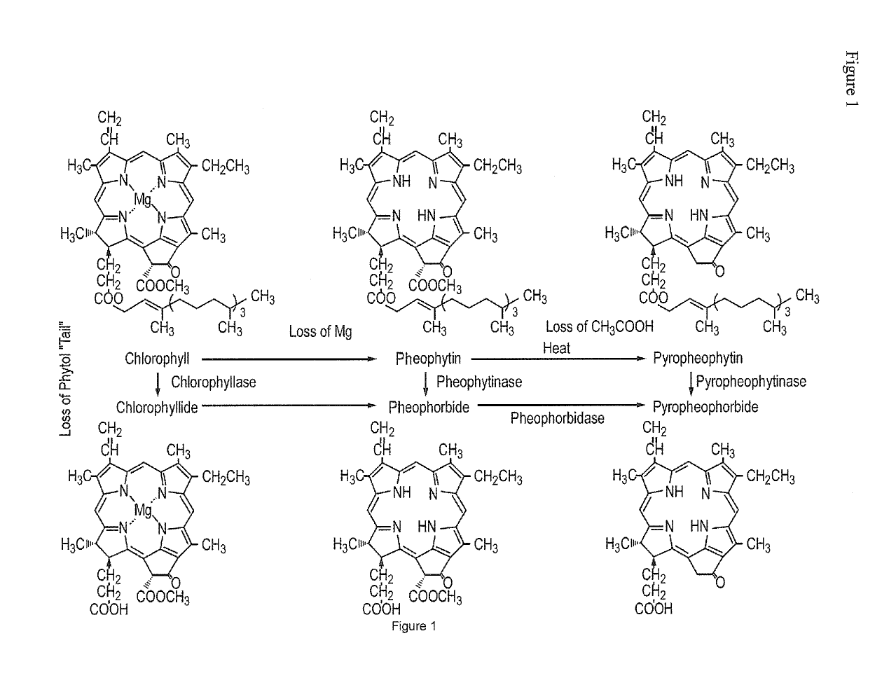

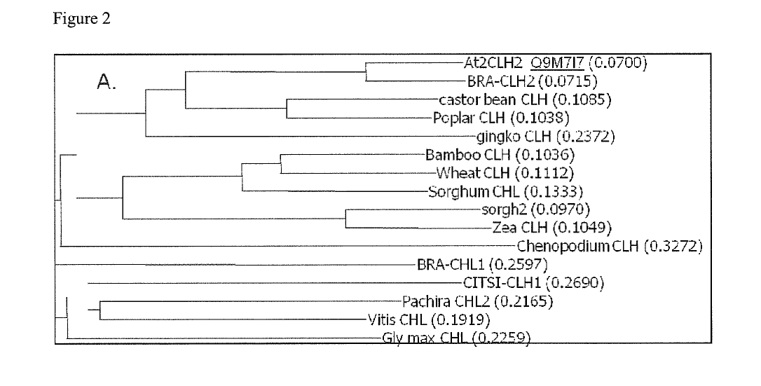

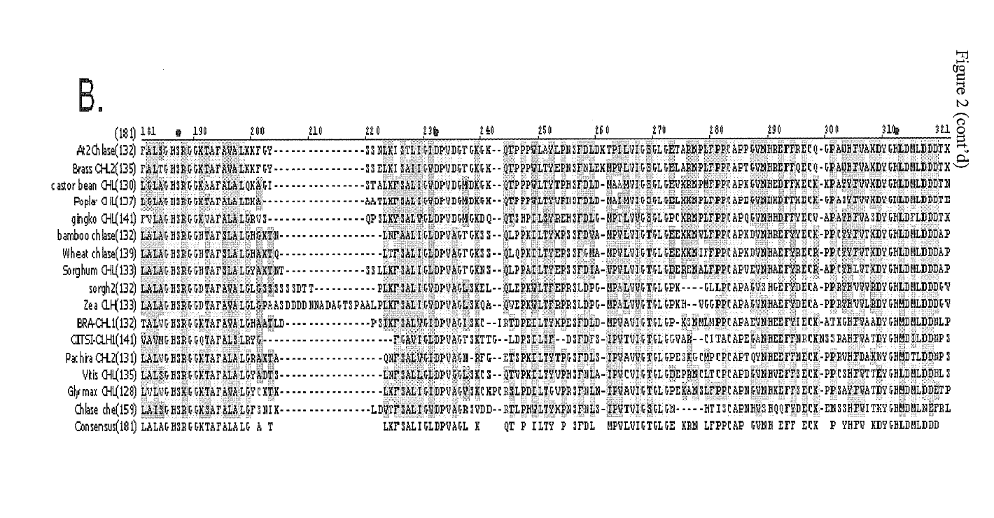

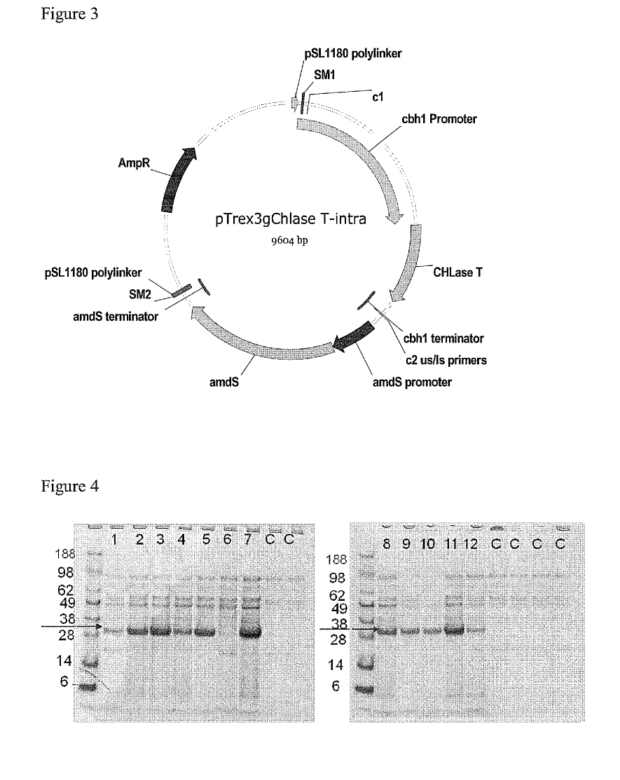

### DETAILED DESCRIPTION

In one aspect, the present invention relates to intracellular expression of enzymes such as chlorophyllases in microbial and/or eukaryotic cells. It has been surprisingly found that intracellular expression in eukaryotes such as fungi rapidly results in a high yield of active enzyme, for example compared to known prokaryotic (bacterial) expression methods. In particular, it has been shown that in eukaryotes such as fungi, intracellular expression is faster and produces a higher enzyme yield than secretion into the extracellular medium.

The fungal cell membrane is surrounded by a thick, tough and rigid cell wall which can hinder extraction of intracellular recombinant products. The cell wall consists of different polymers (chitin, glucans and mannoproteins) surrounding the plasma membrane. Extraction techniques may be considered to involve harsh conditions, which could potentially damage desired recombinant products (reduce their functionality) or reduce the recovery and yield of the recombinant protein (reducing industrial efficiency and raising cost in use of the desirable recombinant products). Moreover, some extraction methods such as enzymatic digestion of the cell walls may be perceived to be expensive, while physical disruption such as bead beating and agitation can cause foaming and sample heating. Sonication may be considered to be sub-optimal due to noise, sample heating and free radical formation damaging the protein of interest.

If a recombinant protein is secreted following expression in a host cell, no cell lysis is required and the enzyme can be recovered directly from the culture medium. For these reasons, secretory expression is often considered to be the preferred method for producing recombinant proteins in microbes such as fungi. In contrast, the present invention surprisingly demonstrates that recombinant chlorophyllases can be produced rapidly and in high yield using intracellular expression in eukaryotes such as fungi, and that the fully functional enzyme can easily be recovered from the cells using simple and straightforward methods.

In one aspect, the present invention relates to a method for producing a recombinant enzyme capable of hydrolyzing chlorophyll or a chlorophyll derivative.

### Chlorophyll and Chlorophyll Derivatives

By “chlorophyll derivative” it is typically meant compounds which comprise both a porphyrin (chlorin) ring and a phytol group (tail), including magnesium-free phytol-containing derivatives such as pheophytin and pyropheophytin. Chlorophyll and (phytol-containing) chlorophyll derivatives are typically greenish in colour, as a result of the porphyrin (chlorin) ring present in the molecule. Loss of magnesium from the porphyrin ring means that pheophytin and pyropheophytin are more brownish in colour than chlorophyll.

The enzymes produced in the present method may hydrolyse chlorophyll and phytol-containing chlorophyll derivatives to cleave the phytol tail from the chlorin ring. Hydrolysis of chlorophyll and chlorophyll derivatives typically results in compounds such as chlorophyllide, pheophorbide and pyropheophorbide which are phytol-free derivatives of chlorophyll. These compounds still contain the colour-bearing porphyrin ring, with chlorophyllide being green and pheophorbide and pyropheophorbide a reddish brown colour.

The chlorophyll or chlorophyll derivative may be either a or b forms. Thus as used herein, the term “chlorophyll” includes chlorophyll a and chlorophyll b. In a similar way both a and b forms are covered when referring to pheophytin, pyropheophytin, chlorophyllide, pheophorbide and pyropheophorbide.

### Enzymes Capable of Hydrolysing Chlorophyll or a Chlorophyll Derivative

The method of the present invention produces an enzyme which is capable of hydrolysing chlorophyll or a chlorophyll derivative. Typically “hydrolyzing chlorophyll or a chlorophyll derivative” means hydrolysing an ester bond in chlorophyll or a (phytol-containing) chlorophyll derivative, e.g. to cleave a phytol group from the chlorin ring in the chlorophyll or chlorophyll derivative. Thus the enzyme typically has an esterase or hydrolase activity. Preferably the enzyme has esterase or hydrolase activity in an oil phase, and optionally also in an aqueous phase.

Thus the enzyme may, for example, be a chlorophyllase, pheophytinase or pyropheophytinase. Preferably, the enzyme is capable of hydrolysing at least one, at least two or all three of chlorophyll, pheophytin and pyropheophytin. In a particularly preferred embodiment, the enzyme has chlorophyllase, pheophytinase and pyropheophytinase activity.

The enzyme produced in the method may be any polypeptide having an activity that can hydrolyse chlorophyll or a chlorophyll derivative. By “enzyme” it is intended to encompass any polypeptide having hydrolytic activity on chlorophyll or a chlorophyll derivative, including e.g. enzyme fragments, etc.

### Enzyme (Chlorophyllase, Pheophytinase or Pyropheophytinase) Activity Assay

Hydrolytic activity on chlorophyll or a chlorophyll derivative may be detected using any suitable assay technique, for example based on an assay described herein. For example, hydrolytic activity may be detected using fluorescence-based techniques. In one suitable assay, a polypeptide to be tested for hydrolytic activity on chlorophyll or a chlorophyll derivative is incubated in the presence of a substrate, and product or substrate levels are monitored by fluorescence measurement. Suitable substrates include e.g. chlorophyll, pheophytin and/or pyropheophytin. Products which may be detected include chlorophyllide, pheophorbide, pyropheophorbide and/or phytol.

Assay methods for detecting hydrolysis of chlorophyll or a chlorophyll derivative are disclosed in, for example, Ali Khamessan et al. (1994), Journal of Chemical Technology and Biotechnology, 60(1), pages 73-81; Klein and Vishniac (1961), J. Biol. Chem. 236: 2544-2547; and Kiani et al. (2006), Analytical Biochemistry 353: 93-98.

Alternatively, a suitable assay may be based on HPLC detection and quantitation of substrate or product levels following addition of a putative enzyme, e.g. based on the techniques described below. In one embodiment, the assay may be performed as described in Hornero-Mendez et al. (2005), Food Research International 38(8-9): 1067-1072. In another embodiment, the following assay may be used:

170 μl mM HEPES, pH 7.0 is added 20 μl 0.3 mM chlorophyll, pheophytin or pyropheophytin dissolved in acetone. The enzyme is dissolved in 50 mM HEPES, pH 7.0. 10 μl enzyme solution is added to 190 μl substrate solution to initiate the reaction and incubated at 40° C. for various time periods. The reaction was stopped by addition of 350 μl acetone. Following centrifugation (2 min at 18,000 g) the supernatant was analyzed by HPLC, and the amounts of (i) chlorophyll and chlorophyllide (ii) pheophytin and pheophorbide or (iii) pyropheophytin and pyropheophorbide determined.

One unit of enzyme activity is defined as the amount of enzyme which hydrolyzes one micromole of substrate (e.g. chlorophyll, pheophytin or pyropheophytin) per minute at 40° C., e.g. in an assay method as described herein.

In preferred embodiments, the enzyme produced in the present method has chlorophyllase, pheophytinase and/or pyropheophytinase activity of at least 1000 U/g, at least 5000 U/g, at least 10000 U/g, or at least 50000 U/g, based on the units of activity per gram of the purified enzyme, e.g. as determined by an assay method described herein.

In a further embodiment, hydrolytic activity on chlorophyll or a chlorophyll derivative may be determined using a method as described in EP10159327.5.

### Chlorophyllases

In one embodiment, the enzyme is capable of hydrolyzing at least chlorophyll. Any polypeptide that catalyses the hydrolysis of a chlorophyll ester bond to yield chlorophyllide and phytol may be produced in the method. For example, a chlorophyllase, chlase or chlorophyll chlorophyllido-hydrolyase or polypeptide having a similar activity (e.g., chlorophyll-chlorophyllido hydrolase 1 or chlase 1, or, chlorophyll-chlorophyllido hydrolase 2 or chlase 2, see, e.g. NCBI P59677-1 and P59678, respectively) may be produced.

In one embodiment the enzyme is a chlorophyllase classified under the Enzyme Nomenclature classification (E.C. 3.1.1.14). In one aspect, the chlorophyllase may be an enzyme as described in WO 0229022 or WO 2006009676. For example, the Arabidopsis thaliana chlorophyllase can be used as described, e.g. in NCBI entry NM_{—}123753. In another embodiment, the chlorophyllase is derived from algae, e.g. from Phaeodactylum tricornutum.

In another embodiment, the chlorophyllase is derived from wheat, e.g. from Triticum spp., especially from Triticum aestivum. In another embodiment, the chlorophyllase is derived from Chlamydomonas spp., especially from Chlamydomonas reinhardtii.

### Pheophytin Pheophorbide Hydrolase

In one embodiment, the enzyme is capable of hydrolyzing pheophytin and pyropheophytin. For example, the enzyme may be pheophytinase or pheophytin pheophorbide hydrolase (PPH), e.g. an enzyme as described in Schelbert et al., The Plant Cell 21:767-785 (2009).

PPH and related enzymes are capable of hydrolyzing pyropheophytin in addition to pheophytin. However PPH is inactive on chlorophyll. As described in Schelbert et al., PPH orthologs are commonly present in eukaryotic photosynthesizing organisms. PPHs represent a defined sub-group of α/β hydrolases which are phylogenetically distinct from chlorophyllases, the two groups being distinguished in terms of sequence homology and substrates.

In specific embodiments of the invention, the enzyme may be any known PPH derived from any species or a functional variant or fragment thereof or may be derived from any known PPH enzyme. For example, in one embodiment, the enzyme is a PPH from Arabidopsis thaliana, (see FIG. 8, NCBI accession no. NP_{—}196884, GenBank ID No. 15240707), or a functional variant or fragment thereof.

In further embodiments, the enzyme may be a PPH derived from any one of the following species: Arabidopsis thaliana, Populus trichocarpa, Vitis vinifera, Oryza sativa, Zea mays, Nicotiana tabacum, Ostreococcus lucimarinus, Ostreococcus taurii, Physcomitrella patens, Phaeodactylum tricornutum, Chlamydomonas reinhardtii, or Micromonas sp. RCC299. For example, the enzyme may be a polypeptide comprising an amino acid sequence, or encoded by a nucleotide sequence, defined in one of the following database entries shown in Table 1, or a functional fragment or variant thereof:

TABLE 1

| Organism | Accession | Genbank ID |
|---|---|---|
| Arabidopsis thaliana | NP_196884 | 15240707 |
| Populus trichocarpa | XP_002314066 | 224106163 |
| Vitis vinifera | CAO40741 | 157350650 |
| Oryza sativa (japonica) | NP_001057593 | 115467988 |
| Zea mays | ACF87407 | 194706646 |
| Nicotiana tabacum | CAO99125 | 156763846 |
| Ostreococcus lucimarinus | XP_001415589 | 145340970 |
| Ostreococcus tauri | CAL50341 | 116000661 |
| Physcomitrella patens | XP_001761725 | 168018382 |
| Phaeodactylum tricornutum | XP_002181821 | 219122997 |
| Chlamydomonas reinhardtii | XP_001702982 | 159490010 |
| Micromonas sp. RCC299 | ACO62405 | 226516410 |

### Variants and Fragments

Functional variants and fragments of known sequences which hydrolyse chlorophyll or a chlorophyll derivative may also be produced in the present invention. By “functional” it is meant that the fragment or variant retains a detectable hydrolytic activity on chlorophyll or a chlorophyll derivative. Typically such variants and fragments show homology to a known chlorophyllase, pheophytinase or pyropheophytinase sequence, e.g. at least about 50%, 60%, 70%, 75%, 80%, 85%, 90%, 95%, 96%, 97%, 98%, 99%, or more sequence identity to a known chlorophyllase, pheophytinase or pyropheophytinase amino acid sequence, e.g. over a region of at least about 10, 20, 30, 50, 100, 200, 300, 500, or 1000 or more residues, or over the entire length of the sequence.

The percentage of sequence identity may be determined by analysis with a sequence comparison algorithm or by a visual inspection. In one aspect, the sequence comparison algorithm is a BLAST algorithm, e.g., a BLAST version 2.2.2 algorithm.

Other enzymes having chlorophyllase, pheophytinase and/or pyropheophytinase activity which may be produced in the present method may be identified by determining the presence of conserved sequence motifs present e.g. in known chlorophyllase, pheophytinase or pyropheophytinase sequences. Polypeptide sequences having suitable activity may be identified by searching genome databases, e.g. the microbiome metagenome database (JGI-DOE, USA), for the presence of these motifs.

### Isolation of Enzyme-Encoding Nucleotide Sequences

In the present method, the enzyme is produced by expression in a eukaryotic host cell using recombinant DNA techniques. Nucleotide sequences encoding polypeptides having chlorophyllase, pheophytinase and/or pyropheophytinase activity may be isolated or constructed and used to express the corresponding polypeptides intracellularly in the host cell.

For example, a genomic DNA and/or cDNA library may be constructed using chromosomal DNA or messenger RNA from an organism which naturally produces the enzyme. If the amino acid sequence of the enzyme is known, labeled oligonucleotide probes may be synthesised and used to identify polypeptide-encoding clones from the genomic library prepared from the organism. Alternatively, a labelled oligonucleotide probe containing sequences homologous to another known polypeptide gene could be used to identify polypeptide-encoding clones. In the latter case, hybridisation and washing conditions of lower stringency are used.

In a yet further alternative, the nucleotide sequence encoding the enzyme may be prepared synthetically by established standard methods, e.g. the phosphoroamidite method described by Beucage S. L. et al (1981) Tetrahedron Letters 22, p 1859-1869, or the method described by Matthes et al (1984) EMBO J. 3, p 801-805. In the phosphoroamidite method, oligonucleotides are synthesised, e.g. in an automatic DNA synthesiser, purified, annealed, ligated and cloned in appropriate vectors.

The nucleotide sequence may be of mixed genomic and synthetic origin, mixed synthetic and cDNA origin, or mixed genomic and cDNA origin, prepared by ligating fragments of synthetic, genomic or cDNA origin (as appropriate) in accordance with standard techniques. Each ligated fragment corresponds to various parts of the entire nucleotide sequence. The DNA sequence may also be prepared by polymerase chain reaction (PCR) using specific primers, for instance as described in U.S. Pat. No. 4,683,202 or in Saiki R K et al (Science (1988) 239, pp 487-491).

The term “nucleotide sequence” as used herein refers to an oligonucleotide sequence or polynucleotide sequence, and variant, homologues, fragments and derivatives thereof (such as portions thereof). The nucleotide sequence may be of genomic or synthetic or recombinant origin, which may be double-stranded or single-stranded whether representing the sense or antisense strand.

Typically, the nucleotide sequence encoding a polypeptide having chlorophyllase, pheophytinase and/or pyropheophytinase activity is prepared using recombinant DNA techniques. However, in an alternative embodiment of the invention, the nucleotide sequence could be synthesised, in whole or in part, using chemical methods well known in the art (see Caruthers M H et al (1980) Nuc Acids Res Symp Ser 215-23 and Horn T et al (1980) Nuc Acids Res Symp Ser 225-232).

### Modification of Enzyme Sequences

Once an enzyme-encoding nucleotide sequence has been isolated, or a putative enzyme-encoding nucleotide sequence has been identified, it may be desirable to modify the selected nucleotide sequence, for example it may be desirable to mutate the sequence in order to prepare an enzyme in accordance with the present invention.

Mutations may be introduced using synthetic oligonucleotides. These oligonucleotides contain nucleotide sequences flanking the desired mutation sites. A suitable method is disclosed in Morinaga et al (Biotechnology (1984)₂, p646-649). Another method of introducing mutations into enzyme-encoding nucleotide sequences is described in Nelson and Long (Analytical Biochemistry (1989), 180, p 147-151).

Instead of site directed mutagenesis, such as described above, one can introduce mutations randomly for instance using a commercial kit such as the GeneMorph PCR mutagenesis kit from Stratagene, or the Diversify PCR random mutagenesis kit from Clontech. EP 0 583 265 refers to methods of optimising PCR based mutagenesis, which can also be combined with the use of mutagenic DNA analogues such as those described in EP 0 866 796. Error prone PCR technologies are suitable for the production of variants of enzymes which hydrolyse chlorophyll and/or chlorophyll derivatives with preferred characteristics. WO0206457 refers to molecular evolution of lipases.

A third method to obtain novel sequences is to fragment non-identical nucleotide sequences, either by using any number of restriction enzymes or an enzyme such as Dnase I, and reassembling full nucleotide sequences coding for functional proteins. Alternatively one can use one or multiple non-identical nucleotide sequences and introduce mutations during the reassembly of the full nucleotide sequence. DNA shuffling and family shuffling technologies are suitable for the production of variants of enzymes with preferred characteristics. Suitable methods for performing ‘shuffling’ can be found in EP0752008, EP1138763, EP1103606. Shuffling can also be combined with other forms of DNA mutagenesis as described in U.S. Pat. No. 6,180,406 and WO 01/34835.

Thus, it is possible to produce numerous site directed or random mutations into a nucleotide sequence, either in vivo or in vitro, and to subsequently screen for improved functionality of the encoded polypeptide by various means. Using in silico and exo mediated recombination methods (see WO 00/58517, U.S. Pat. No. 6,344,328, U.S. Pat. No. 6,361,974), for example, molecular evolution can be performed where the variant produced retains very low homology to known enzymes or proteins. Such variants thereby obtained may have significant structural analogy to known chlorophyllase, pheophytinase or pyropheophytinase enzymes, but have very low amino acid sequence homology.

As a non-limiting example, in addition, mutations or natural variants of a polynucleotide sequence can be recombined with either the wild type or other mutations or natural variants to produce new variants. Such new variants can also be screened for improved functionality of the encoded polypeptide.

The application of the above-mentioned and similar molecular evolution methods allows the identification and selection of variants of the enzymes of the present invention which have preferred characteristics without any prior knowledge of protein structure or function, and allows the production of non-predictable but beneficial mutations or variants. There are numerous examples of the application of molecular evolution in the art for the optimisation or alteration of enzyme activity, such examples include, but are not limited to one or more of the following: optimised expression and/or activity in a host cell or in vitro, increased enzymatic activity, altered substrate and/or product specificity, increased or decreased enzymatic or structural stability, altered enzymatic activity/specificity in preferred environmental conditions, e.g. temperature, pH, substrate.

As will be apparent to a person skilled in the art, using molecular evolution tools an enzyme may be altered to improve the functionality of the enzyme. Suitably, a nucleotide sequence encoding an enzyme (e.g. a chlorophyllase, pheophytinase and/or pyropheophytinase) produced in the present method may encode a variant enzyme, i.e. the variant enzyme may contain at least one amino acid substitution, deletion or addition, when compared to a parental enzyme. Variant enzymes retain at least 1%, 2%, 3%, 5%, 10%, 15%, 20%, 30%, 40%, 50%, 60%, 70%, 80%, 90%, 95%, 97%, or 99% identity with the parent enzyme. Suitable parent enzymes may include any enzyme with hydrolytic activity on chlorophyll and/or a chlorophyll derivative.

### Sequence Comparison

Here, the term “homologue” means an entity having a certain homology with the subject amino acid sequences and the subject nucleotide sequences. Here, the term “homology” can be equated with “identity”. The homologous amino acid sequence and/or nucleotide sequence should provide and/or encode a polypeptide which retains the functional activity and/or enhances the activity of the enzyme.

In the present context, a homologous sequence is taken to include an amino acid sequence which may be at least 75, 85 or 90% identical, preferably at least 95 or 98% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, a homologous sequence is taken to include a nucleotide sequence which may be at least 75, 85 or 90% identical, preferably at least 95 or 98% identical to a nucleotide sequence encoding a polypeptide of the present invention (the subject sequence). Typically, the homologues will comprise the same sequences that code for the active sites etc. as the subject sequence. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences. % homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons.

Calculation of maximum % homology therefore firstly requires the production of an optimal alignment, taking into consideration gap penalties. A suitable computer program for carrying out such an alignment is the Vector NTI Advance™ 11 (Invitrogen Corp.). Examples of other software that can perform sequence comparisons include, but are not limited to, the BLAST package (see Ausubel et al 1999 Short Protocols in Molecular Biology, 4th Ed—Chapter 18), and FASTA (Altschul et al 1990 J. Mol. Biol. 403-410). Both BLAST and FASTA are available for offline and online searching (see Ausubel et al 1999, pages 7-58 to 7-60). However, for some applications, it is preferred to use the Vector NTI Advance™ 11 program. A new tool, called BLAST 2 Sequences is also available for comparing protein and nucleotide sequence (see FEMS Microbiol Lett 1999 174(2): 247-50; and FEMS Microbiol Lett 1999 177(1): 187-8.).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. Vector NTI programs generally use either the public default values or a custom symbol comparison table if supplied (see user manual for further details). For some applications, it is preferred to use the default values for the Vector NTI Advance™ 11 package.

Alternatively, percentage homologies may be calculated using the multiple alignment feature in Vector NTI Advance™ 11 (Invitrogen Corp.), based on an algorithm, analogous to CLUSTAL (Higgins DG and Sharp PM (1988), Gene 73(1), 237-244). Once the software has produced an optimal alignment, it is possible to calculate % homology, preferably % sequence identity. The software typically does this as part of the sequence comparison and generates a numerical result.

Should Gap Penalties be used when determining sequence identity, then preferably the default parameters for the programme are used for pairwise alignment. For example, the following parameters are the current default parameters for pairwise alignment for BLAST 2:

| FOR BLAST2 | DNA | PROTEIN |
|---|---|---|
| EXPECT THRESHOLD | 10 | 10 |
| WORD SIZE | 11 | 3 |
| SCORING PARAMETERS | | |
| Match/Mismatch Scores | 2, −3 | n/a |
| Matrix | n/a | BLOSUM62 |
| Gap Costs | Existence: 5 | Existence: 11 |
| | Extension: 2 | Extension: 1 |

In one embodiment, preferably the sequence identity for the nucleotide sequences and/or amino acid sequences may be determined using BLAST2 (blastn) with the scoring parameters set as defined above.

For the purposes of the present invention, the degree of identity is based on the number of sequence elements which are the same. The degree of identity in accordance with the present invention for amino acid sequences may be suitably determined by means of computer programs known in the art such as Vector NTI Advance™ 11 (Invitrogen Corp.). For pairwise alignment the scoring parameters used are preferably BLOSUM62 with Gap existence penalty of 11 and Gap extension penalty of 1.

Suitably, the degree of identity with regard to a nucleotide sequence is determined over at least 20 contiguous nucleotides, preferably over at least 30 contiguous nucleotides, preferably over at least 40 contiguous nucleotides, preferably over at least 50 contiguous nucleotides, preferably over at least 60 contiguous nucleotides, preferably over at least 100 contiguous nucleotides. Suitably, the degree of identity with regard to a nucleotide sequence may be determined over the whole sequence.

### Amino Acid Mutations

The sequences may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar—uncharged | C S T M |
| | | N Q |
| | Polar —charged | D E |
| | | K R |
| AROMATIC | | H F W Y |

The present invention also encompasses homologous substitution (substitution and replacement are both used herein to mean the interchange of an existing amino acid residue, with an alternative residue) that may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine. Replacements may also be made by unnatural amino acids.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

### Nucleotide Sequences

Nucleotide sequences for use in the present invention or encoding a polypeptide having the specific properties defined herein may include within them synthetic or modified nucleotides. A number of different types of modification to oligonucleotides are known in the art. These include methylphosphonate and phosphorothioate backbones and/or the addition of acridine or polylysine chains at the 3′ and/or 5′ ends of the molecule. For the purposes of the present invention, it is to be understood that the nucleotide sequences described herein may be modified by any method available in the art. Such modifications may be carried out in order to enhance the in vivo activity or life span of nucleotide sequences.

The present invention also encompasses the use of nucleotide sequences that are complementary to the sequences discussed herein, or any derivative, fragment or derivative thereof. If the sequence is complementary to a fragment thereof then that sequence can be used as a probe to identify similar coding sequences in other organisms etc.

Polynucleotides which are not 100% homologous to the sequences of the present invention but fall within the scope of the invention can be obtained in a number of ways. Other variants of the sequences described herein may be obtained for example by probing DNA libraries made from a range of individuals, for example individuals from different populations. In addition, other viral/bacterial, or cellular homologues particularly cellular homologues found in plant cells, may be obtained and such homologues and fragments thereof in general will be capable of selectively hybridising to the sequences shown in the sequence listing herein. Such sequences may be obtained by probing cDNA libraries made from or genomic DNA libraries from other plant species, and probing such libraries with probes comprising all or part of any one of the sequences in the attached sequence listings under conditions of medium to high stringency. Similar considerations apply to obtaining species homologues and allelic variants of the polypeptide or nucleotide sequences of the invention.

Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used.

The primers used in degenerate PCR will contain one or more degenerate positions and will be used at stringency conditions lower than those used for cloning sequences with single sequence primers against known sequences.

Alternatively, such polynucleotides may be obtained by site directed mutagenesis of characterised sequences. This may be useful where for example silent codon sequence changes are required to optimise codon preferences for a particular host cell in which the polynucleotide sequences are being expressed. Other sequence changes may be desired in order to introduce restriction polypeptide recognition sites, or to alter the property or function of the polypeptides encoded by the polynucleotides.

Polynucleotides (nucleotide sequences) of the invention may be used to produce a primer, e.g. a PCR primer, a primer for an alternative amplification reaction, a probe e.g. labelled with a revealing label by conventional means using radioactive or non-radioactive labels, or the polynucleotides may be cloned into vectors. Such primers, probes and other fragments will be at least 15, preferably at least 20, for example at least 25, 30 or 40 nucleotides in length, and are also encompassed by the term polynucleotides of the invention as used herein.

Polynucleotides such as DNA polynucleotides and probes according to the invention may be produced recombinantly, synthetically, or by any means available to those of skill in the art. They may also be cloned by standard techniques.

In general, primers will be produced by synthetic means, involving a stepwise manufacture of the desired nucleic acid sequence one nucleotide at a time. Techniques for accomplishing this using automated techniques are readily available in the art.

Longer polynucleotides will generally be produced using recombinant means, for example using a PCR (polymerase chain reaction) cloning techniques. This will involve making a pair of primers (e.g. of about 15 to 30 nucleotides) flanking a region of the pyropheophytinase sequence which it is desired to clone, bringing the primers into contact with mRNA or cDNA obtained from a plant cell, performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

### Nucleic Acid Constructs

In one embodiment of the present invention, the method includes a step of introducing a nucleic acid construct (e.g. an expression vector) encoding the recombinant enzyme into the host cell. Thus the invention further provides a nucleic acid construct (e.g. a eukaryotic expression vector) comprising a nucleic acid sequence encoding an enzyme capable of hydrolysing chlorophyll or a chlorophyll derivative, operably linked to one or more control sequences which direct the expression of the coding sequence in a eukaryotic host cell.

By “eukaryotic expression vector” it is meant that the vector is capable of directing expression of the recombinant enzyme in a eukaryotic host cell, preferably a fungal host cell. In other words, the vector typically contains suitable regulatory and/or control sequences which are functional in eukaryotic (e.g. fungal) cells.

For example, the control sequence may be an appropriate promoter sequence, e.g. a nucleotide sequence which is recognized by a eukaryotic (e.g. fungal) host cell for expression of a polynucleotide encoding the enzyme. The promoter sequence contains transcriptional control sequences which mediate the expression of the polypeptide.

The promoter may be any nucleotide sequence which shows transcriptional activity in the eukaryotic (e.g. fungal) host cell including mutant, truncated, and hybrid promoters, and may be obtained from genes encoding extracellular or intracellular polypeptides either homologous or heterologous to the host cell.

Examples of suitable promoters for directing the transcription of the nucleic acid constructs of the present invention in a filamentous fungal host cell are promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, Fusarium venenatum amyloglucosidase (WO 00/56900), Fusarium venenatum Daria (WO 00/56900), Fusarium venenatum Quinn (WO 00/56900), Fusarium oxysporum trypsin-like protease (WO 96/00787), Trichoderma reesei beta-glucosidase, Trichoderma reesei cellobiohydrolase I, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a hybrid of the promoters from the genes for Aspergillus niger neutral alpha-amylase and Aspergillus oryzae triose phosphate isomerase); and mutant, truncated, and hybrid promoters thereof. In a preferred embodiment, the promoter is derived from the Trichoderma reesei cbh1 gene.

In a yeast host, useful promoters may be obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1,ADH2/GAP), Saccharomyces cerevisiae triose phosphate isomerase (TPI), Saccharomyces cerevisiae metallothionine (CUP 1), and Saccharomyces cerevisiae 3-phosphoglycerate kinase. Other useful promoters for yeast host cells are described by Romanos et al., 1992, Yeast 8: 423-488.

The control sequence may also be a suitable transcription terminator sequence, i.e. a sequence recognized by a eukaryotic (e.g. fungal) host cell to terminate transcription. Typically the terminator sequence is operably linked to the 3′ terminus of the nucleotide sequence encoding the polypeptide. Any terminator which is functional in a eukaryotic (e.g fungal) host cell may be used in the present invention.

Preferred terminators for filamentous fungal host cells are obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. In a preferred embodiment, the terminator is derived from the Trichoderma reesei cbh1 gene.

Preferred terminators for yeast host cells are obtained from the genes for Saccharomyces cerevisiae enolase, Saccharomyces cerevisiae cytochrome C(CYC1), and Saccharomyces cerevisiae glyceraldehyde-3-phosphate dehydrogenase. Other useful terminators for yeast host cells are described by Romanos et al., 1992, supra.

The control sequence may also be a suitable leader sequence, i.e. a nontranslated region of an mRNA which is important for translation by the host cell. Typically the leader sequence is operably linked to the 5′ terminus of the nucleotide sequence encoding the polypeptide. Any leader sequence that is functional in a eukaryotic (e.g. fungal) cell may be used in the present invention.

Preferred leaders for filamentous fungal host cells are obtained from the genes for Aspergillus oryzae TAKA amylase and Aspergillus nidulans phosphate isomerase. Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, i.e. a sequence operably linked to the 3′ terminus of the nucleotide sequence and which, when transcribed, is recognized by the host cell as a signal to add polyadenosine residues to transcribed mRNA. Any polyadenylation sequence which is functional in a eukaryotic (e.g. fungal) cell may be used in the present invention.

Preferred polyadenylation sequences for filamentous fungal host cells are obtained from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase.

Useful polyadenylation sequences for yeast host cells are described by Guo and Sherman, 1995, Molecular Cellular Biology 15: 5983-5990.

The control sequence may also be a propeptide coding region that codes for an amino acid sequence positioned at the amino terminus of a polypeptide. The resultant polypeptide is known as a proenzyme or propolypeptide (or a zymogen in some cases). A propolypeptide is generally inactive and can be converted to a mature active polypeptide by catalytic or autocatalytic cleavage of the propeptide from the propolypeptide.

The propeptide coding region may be obtained from the genes for Saccharomyces cerevisiae alpha-factor, Rhizomucor miehei aspartic proteinase, Myceliophthora thermophila laccase (WO 95/33836), Humicola insolens cutinase (WO 2005121333), Candida albicans lipase B (CLB) or Candida antarctica lipase B (CLB′).

The expression vector typically directs intracellular expression of the recombinant enzyme. By this it is meant that when introduced into a eukaryotic host cell, the vector leads to cytoplasmic translation of a recombinant enzyme which is not targeted for secretion, e.g. the recombinant enzyme accumulates predominantly within the cell (rather than being secreted or becoming membrane-bound). In some embodiments, some of the recombinant enzyme may be present outside the cell, for example due to the enzyme diffusing across the cell membrane or due to partial cell lysis. However, “intracellular expression” typically refers to embodiments where the encoded enzyme is not targeted for extracellular secretion via the cell's intrinsic secretory pathway.

Thus the expression vector typically does not comprise a signal peptide coding region, such that the encoded recombinant enzyme is expressed without a signal peptide. A signal peptide is an amino acid sequence linked to the amino terminus of a polypeptide which directs the encoded polypeptide into the cell's secretory pathway. Thus according to embodiments of the present invention, absence of a signal peptide coding region in the expression vector leads to intracellular expression of the recombinant enzyme.

The 5′ end of the coding sequence for the enzyme may inherently contain a signal peptide coding region naturally linked in translation reading frame with the segment of the coding region which encodes the enzyme. In some embodiments, the signal peptide coding region may be deleted from the natural sequence before insertion into the expression vector.

In some embodiments, the expression vector further comprises regulatory sequences which allow the regulation of the expression of the polypeptide relative to the growth of the host cell. Examples of regulatory systems are those which cause the expression of the gene to be turned on or off in response to a chemical or physical stimulus, including the presence of a regulatory compound. In yeast, the ADH2 system or GAL1 system may be used. In filamentous fungi, the TAKA alpha-amylase promoter, Aspergillus niger glucoamylase promoter, and Aspergillus oryzae glucoamylase promoter may be used as regulatory sequences.

Other examples of regulatory sequences are those which allow for gene amplification. In eukaryotic systems, these include the dihydrofolate reductase gene which is amplified in the presence of methotrexate, and the metallothionein genes which are amplified with heavy metals. In these cases, the nucleotide sequence encoding the enzyme would be operably linked with the regulatory sequence.

Typically the expression vector comprises an enzyme-encoding sequence, a promoter, and transcriptional and translational stop signals. The various nucleic acids and control sequences described above may be joined together to produce a recombinant expression vector which may include one or more convenient restriction sites to allow for insertion or substitution of the nucleotide sequence encoding the enzyme at such sites. In creating the expression vector, the coding sequence is located in the vector so that the coding sequence is operably linked with the appropriate control sequences for expression.

The recombinant expression vector may be any vector (e.g., a plasmid or virus) which can be conveniently subjected to recombinant DNA procedures and can bring about expression of the nucleotide sequence. The choice of the vector will typically depend on the compatibility of the vector with the host cell into which the vector is to be introduced. The vectors may be linear or closed circular plasmids.

The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication.

Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

The vectors of the present invention preferably contain one or more selectable markers which permit easy selection of transformed cells. A selectable marker is a gene the product of which provides for biocide or viral resistance, resistance to heavy metals, prototrophy to auxotrophs, and the like.

Suitable markers for yeast host cells are ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyl-transferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof.

Preferred for use in an Aspergillus or Trichoderma cell are the amdS and pyrG genes of Aspergillus nidulans or Aspergillus oryzae and the bar gene of Streptomyces hygroscopicus.

The vectors of the present invention may contain an element(s) that permits integration of the vector into the host cell's genome or autonomous replication of the vector in the cell independent of the genome. For integration into the host cell genome, the vector may rely on the polynucleotide's sequence encoding the enzyme or any other element of the vector for integration into the genome by homologous or nonhomologous recombination.

Alternatively, the vector may contain additional nucleotide sequences for directing integration by homologous recombination into the genome of the host cell at a precise location(s) in the chromosome(s). To increase the likelihood of integration at a precise location, the integrational elements should preferably contain a sufficient number of nucleic acids, such as 100 to 10,000 base pairs, preferably 400 to 10,000 base pairs, and most preferably 800 to 10,000 base pairs, which have a high degree of identity with the corresponding target sequence to enhance the probability of homologous recombination.

The integrational elements may be any sequence that is homologous with the target sequence in the genome of the eukaryotic (e.g. fungal) host cell. Furthermore, the integrational elements may be non-encoding or encoding nucleotide sequences. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication which functions in a cell. The term “origin of replication” or “plasmid replicator” is defined herein as a nucleotide sequence that enables a plasmid or vector to replicate in vivo.

Examples of origins of replication for use in a yeast host cell are the 2 micron origin of replication, ARS1, ARS4, the combination of ARS1 and CEN3, and the combination of ARS4 and CEN6. Examples of origins of replication useful in a filamentous fungal cell are AMA1 and ANSI (Gems et al., 1991, Gene 98:61-67; Cullen et al., 1987, Nucleic Acids Research 15: 9163-9175; WO 00/24883). Isolation of the AMA1 gene and construction of plasmids or vectors comprising the gene can be accomplished according to the methods disclosed in WO 00/24883.

More than one copy of an enzyme-encoding sequence may be inserted into the host cell to increase production of the gene product. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art.

### Eukaryotic Host Cells

In embodiments of the present invention, a eukaryotic host cell is transformed with a nucleotide sequence encoding a recombinant enzyme, e.g. as described above. Typically a vector comprising an enzyme-encoding sequence is introduced into a host cell so that the vector is maintained as a chromosomal integrant or as a self-replicating extra-chromosomal vector as described earlier. The term ‘host cell’ encompasses any progeny of a parent cell that is not identical to the parent cell due to mutations that occur during replication.

The eukaryotic host cell is, preferably, selected from the group consisting of a mammalian cell, an insect cell, a plant cell and a fungal cell. Most preferably the eukaryotic host cell is derived from fungi, i.e. the host cell is a fungal cell.

Chlorophyllases are naturally expressed in plants, but the level of chlorophyllase expression in plants is low and tightly regulated during development. Plant cells are less preferred as a host for production of recombinant chlorophyllases because the enzyme is involved in degreening and senescence.

“Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra).

In one embodiment, the fungal host cell is a yeast cell. Yeasts include ascosporogenous yeast (Endomycetales), basidiosporogenous yeast, and yeast belonging to the Fungi Imperfecti (Blastomycetes). Yeast may be defined as described in Biology and Activities of Yeast (Skinner, F. A., Passmore, S. M., and Davenport, R. R., eds, Soc. App. Bacteriol. Symposium Series No. 9, 1980). For example, the host cell may be selected from the genera Candida, Hansenula, Kluyveromyces, Pichia, Saccharomyces, Schizosaccharomyces, or Yarrowia. More specifically, the yeast host cell may be a Saccharomyces carlsbergensis, Saccharomyces cerevisiae, Saccharomyces diastaticus, Saccharomyces douglasii, Saccharomyces kluyveri, Saccharomyces norbensis or Saccharomyces oviformis, Kluyveromyces lactis, Yarrowia lipolytica or Pichia pastoris cell.

In another embodiment, the fungal host cell is a filamentous fungal cell. Filamentous fungi include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. In some embodiments, host cell is derived from a genus selected from Acremonium, Aspergillus, Aureobasidium, Bjerkandera, Ceriporiopsis, Coprinus, Coriolus, Cryptococcus, Filobasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma.

In some embodiments, the host cell is selected from an Aspergillus awamori, Aspergillus fumigatus, Aspergillus foetidus, Aspergillus japonicus, Aspergillus nidulans, Aspergillus niger, Aspergillus oryzae, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum Bjerkandera adusta, Ceriporiopsis aneirina, Chrysosporium lucknowense, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Coprinus cinereus, Coriolus hirsutus, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Most preferably the host cell is from Trichoderma reesei.

Fungal cells may be transformed by a process involving protoplast formation, transformation of the protoplasts, and regeneration of the cell wall in a manner known per se. Alternatively, fungi may be transformed by electroporation or biolistic methods using spores.

Suitable procedures for transformation of Aspergillus and Trichoderma host cells are described in EP 238 023 and Yelton et al., 1984, Proceedings of the National Academy of Sciences USA 81: 1470-1474. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75: 1920.

### Methods of Production

The present method may be used to produce the recombinant enzyme. The method may comprise steps of (a) cultivating the eukaryotic host cell under conditions conducive for production of the enzyme; and (b) recovering the enzyme.

In the production methods of the present invention, the cells are cultivated in a nutrient medium suitable for production of the polypeptide using methods well known in the art. For example, the cell may be cultivated by shake flask cultivation, and small-scale or large-scale fermentation (including continuous, batch, fed-batch, or solid state fermentations) in laboratory or industrial fermentors performed in a suitable medium and under conditions allowing the polypeptide to be expressed and/or isolated.

The cultivation takes place in a suitable nutrient medium comprising carbon and nitrogen sources and inorganic salts, using procedures known in the art. Suitable media are available from commercial suppliers or may be prepared according to published compositions (e.g. in catalogues of the American Type Culture Collection).

### Cell Lysis and Recovery of Intracellular Chlorophyllases

Since the enzyme is produced intracellularly in the host cells, the method of the present invention typically comprises a step of lysing the host cells and recovering the recombinant enzyme from the lysate. This step may be performed using methods such as enzymatic digestion, physical disruption (e.g. bead beating and agitation), sonication, homogenization and/or freeze/thaw cycles. Various methods for cell lysis including heat treatment and enzymatic methods are disclosed, for example, in U.S. Pat. No. 4,601,986, U.S. Pat. No. 4,299,858 and U.S. Pat. No. 3,816,260.

In one preferred embodiment, the host cell (e.g. a filamentous fungal cell such as Trichoderma and Aspergillus mycelia) is lysed using a detergent, e.g. a non-ionic surfactant. For example, the cell lysis step may be performed using commercially available reagents or kits, e.g. Cellytic™ Y Cell Lysis Reagent for yeast cells (available from Sigma-Aldrich Co., St. Louis, Mo., cat. no. C4482).

Nonionic surfactants include carboxylic acid esters, such as glycerol esters and polyoxyethylene esters; anhydrosorbitol esters, such as ethoxylated anhydrosorbitol esters; polyoxyethylene surfactants, such as alcohol ethoxylates and alkylphenol ethoxylates; natural ethoxylated fats, oils and waxes; glycol esters of fatty acids; alkyl polyglycosides; carboxylic amides, such as diethanolamine condensates, monoalkanolamine condensates including coco, lauric, oleic, and stearic monoethanolaimdes and monoisopropanolamides, polyoxyethylene fatty acid amides; fatty acid glucamides; and polyoxyalkylene block copolymers. In a preferred embodiment, the non-ionic surfactant comprises dodecyl trimethyl ammonium bromide (DTAB).

In a further embodiment, the host cell may be killed and/or subsequently lysed using an organic acid treatment. Suitable organic acids include e.g. benzoic acid, sorbic acid, acetic acid, citric acid, propanoic acid and formic acid. In some embodiments an inorganic acid such as sulphuric acid may also be used. In one embodiment, one or more of the above acids may be used to lower the pH of the medium comprising the host cell to, for example, less than pH 5, e.g. pH 3 to 5. Typically the host cell may be exposed to the organic acid at reduced pH for 12 hours to 5 days, e.g. about 24 hours to about 3 hours, at a temperature of 20° C. to 40° C., e.g. about 30° C. In some embodiments, treatment with organic acids results in cell killing, and a subsequent step (e.g. heat treatment step) may be used in order to produce cell lysis. Methods for cell kill without cell lysis using organic acids are disclosed in, for example, U.S. Pat. No. 5,801,034 in connection with secreted proteins. Similar methods may be used herein to recover the intracellular enzyme provided that the cells are subsequently lysed.

The use of a detergent or organic acid treatment can be combined with selected conditions of pH, temperature, buffer composition and ionic strength in order to favour cell lysis. A skilled person can select suitable conditions, which may optionally be combined with homogenization to deliver large scale product recovery. Detergents can advantageously be used to extract recombinant chlorophyllase and can be applied to whole cell broth. Detergents, when used in combination with heat treatment, result in cell lysis as well as selective recovery of chlorophyllases, most of which are heat stable enzymes. The selective release of chlorophyllases is crucial for improving product economics and downstream operations.

The resulting enzyme may be recovered using methods known in the art. For example, the enzyme may be recovered from the nutrient medium by conventional procedures including, but not limited to, centrifugation, filtration, extraction, spray-drying, evaporation, or precipitation.

The enzymes may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989).

The enzymes may be detected using methods known in the art that are specific for the enzyme. These detection methods may include use of specific antibodies, formation of an enzyme product, or disappearance of an enzyme substrate. For example, an enzyme assay as described above may be used to determine the activity of the polypeptide as described herein.

The invention will now be described by way of example only, with reference to the following non-limiting embodiments.

### EXAMPLES

### Example 1

### Identification of Chlorophyllase Genes with Sequence Identity/Similarity to Known Chlorophyllases from Plants

Experiments were conducted to identify genes encoding known and putative chlorophyllases in published sequence databases. The genes and deduced amino acid sequences of several functionally characterized chlorophyllases are known. Some of these known chlorophyllases have been expressed in E. coli alone or as fusion proteins (thioredoxin or maltose binding protein fusions). The chlorophyllases from different plants such as Arabidopsis thaliana (AtCLH1, AtCLH2), Brassica oleracea, (CLH1, CHL2, CRL3), different Citrus species, Gingko biloba, Triticum aestivum, Chenopodium album have been isolated, expressed in E. coli and biochemically characterised. The known chlorophyllase sequences were used as queries in BLAST analyses on the non-redundant (nr) protein database of the National Center for Biotechnology Information (NCBI). Several putative uncharacterized chlorophyllases were identified from different plants.

Table 1 shows the amino acid sequence identity of the known chlorophyllase from Arabidopsis thaliana (AtCLH2) with different known and putative plant chlorophyllases. Sequence identity of 36% to as high as 86% was observed. AtCLH2 has 95% sequence identity to another chlorophyllase, from Arabidopsis lyrata (Accession no. D7MNK2)(not shown). However, chlorophyllases from Brassica oleracea CLH2 (Q8GTM3) shows high sequence identity (86%), while another known Brassica oleracea CLH1 (Q8GTM4) has sequence identity of 44% to the Arabidopsis thaliana CLH2. A wide range of sequence identity/similarity exists between different known and putative plant chlorophyllases.

TABLE 1
Pairwise identity of the amino acid sequence between Arabidopsis thaliana chlorophyllase (AtCLH2),
Accession no. Q9M717) and other plant chlorophyllases.

| | Q9M717 | Q8GTM3 | Xp002517075 | B9HUR3 | Q7Y0K5 | FP092915 | BT009214 | C5YN66 |
|---|---|---|---|---|---|---|---|---|
| Q9M717 | 100^{a} | 86 | 63 | 62 | 54 | 47 | 45 | 47 |
| Q8GTM3 | | 100 | 61 | 62 | 53 | 47 | 46 | 47 |
| Xp002517075 | | | 100 | 79 | 56 | 48 | 47 | 48 |
| B9HUR3 | | | | 100 | 55 | 49 | 47 | 48 |
| Q7Y0K5 | | | | | 100 | 47 | 46 | 45 |
| FP092915 | | | | | | 100 | 78 | 74 |
| BT009214 | | | | | | | 100 | 72 |
| C5YN66 | | | | | | | | 100 |
| C5X5Z9 | | | | | | | | |
| C0PCY5 | | | | | | | | |
| Q8GTM4 | | | | | | | | |
| Q9MV14- | | | | | | | | |
| C1JZ62 | | | | | | | | |
| XP002273926 | | | | | | | | |
| BAF43704 | | | | | | | | |
| Q9LE89 | | | | | | | | |

| | C5X5Z9 | C0PCY5 | Q8GTM4 | Q9MV14 | C1JZ62 | XP002273926 | BAF43704 | Q9LE89 |
|---|---|---|---|---|---|---|---|---|
| Q9M717 | 43 | 41 | 44 | 42 | 46 | 50 | 46 | 36 |
| Q8GTM3 | 45 | 42 | 43 | 41 | 46 | 49 | 45 | 38 |
| Xp002517075 | 44 | 44 | 44 | 41 | 46 | 52 | 45 | 41 |
| B9HUR3 | 46 | 45 | 44 | 43 | 45 | 55 | 49 | 40 |
| Q7Y0K5 | 40 | 42 | 44 | 40 | 47 | 49 | 46 | 35 |
| FP092915 | 52 | 51 | 46 | 42 | 50 | 52 | 48 | 40 |
| BT009214 | 49 | 50 | 46 | 43 | 51 | 52 | 48 | 39 |
| C5YN66 | 50 | 49 | 45 | 43 | 44 | 51 | 47 | 40 |
| C5X5Z9 | 100 | 80 | 42 | 40 | 41 | 47 | 46 | 37 |
| C0PCY5 | | 100 | 41 | 39 | 40 | 45 | 46 | 38 |
| Q8GTM4 | | | 100 | 44 | 50 | 50 | 51 | 41 |
| Q9MV14- | | | | 100 | 51 | 51 | 48 | 38 |
| C1JZ62 | | | | | 100 | 59 | 53 | 42 |
| XP002273926 | | | | | | 100 | 57 | 45 |
| BAF43704 | | | | | | | 100 | 44 |
| Q9LE89 | | | | | | | | 100 |

^{a}Percentage of identity was calculated using a ClustalW multiple alignment program with a BLOSUM62 score matrix.

Alignment of the different plant chlorophyllases that were expressed in a fungal expression host showed conserved blocks around the active site residues identified as the catalytic triad: serine (SER), aspartic acid (ASP), and histidine (HIS). The active site residues have been previously identified by site directed mutagenesis of BoCLH2 (J Agric Food Chem. 2010 Aug. 11; 58(15):8651-7). Sequence alignment of both known putative uncharacterized chlorophyllases showed the presence of conserved blocks around the active site residues. Three signature motifs: GHSXGG (SEQ ID No:36), DPVXG (SEQ ID NO:37), YGHXD (SEQ ID NO:38) could be used to identify new members of the chlorophyllase superfamily either by searching sequenced genome databases, screening metagenomic libraries or by using these amino acids as degenerate oligonucleotide primer/probes in a PCR to identify new genes present in different chlorophyll containing organisms such as plants, algae and chlorophyll containing bacteria. The GHSXGG motif contains the active site serine residue which is a motif common to lipolytic enzymes (lipases) but the 2 other motifs, DPVXG, YGHXD containing the active sites aspartate and histidine, are unique to chlorophyllases. These 3 signature motifs when combined can be used as a diagnostic tool to identify new chlorophyllase candidates from plants, algae and bacteria.

### Example 2

### Cloning and Expression of Chlorophyllases from Plants (Table 1) and Algae in Trichoderma reesei

Expression vectors for production of chlorophyllases from different plants and algae (Chlamydomonas) in fungi (Trichoderma reesei) were made by recombining GATEWAY® entry vector pDONR 221 (Invitrogen, Corp. Carlsbad, Calif., USA) containing synthetic genes encoding each of the plant (Table 2) and algae chlorophyllases with the T. reesei GATEWAY® destination vector pTrex3G (U.S. Pat. No. 7,413,879). The destination vector pTrex3g is based on the E. coli vector pSL1180 (Pharmacia, Inc., Piscataway, N.J., USA) which is a pUC118 phagemid-based vector (Brosius, J. (1989), DNA 8:759) with an extended multiple cloning site containing 64 hexamer restriction enzyme recognition sequences. This plasmid was designed as a Gateway destination vector (Hartley et al. (2000) Genome Research 10:1788-95) to allow insertion using Gateway technology (Invitrogen) of a desired open reading frame between the promoter and terminator regions of the T. reesei cbh1 gene. It also contains the Aspergillus nidulans amdS gene for use as a selective marker in transformation of T. reesei. The pTrex3g is 10.3 kb in size and inserted into the polylinker region of pSL1180 are the following segments of DNA: a) a 2.2 bp segment of DNA from the promoter region of the T. reesei cbh1 gene; b) the 1.7 kb Gateway reading frame A cassette acquired from Invitrogen that includes the attR1 and attR2 recombination sites at either end flanking the chloramphenicol resistance gene (CmR) and the ccdB gene; c) a 336 bp segment of DNA from the terminator region of the T. reesei cbh1 gene; and d) a 2.7 kb fragment of DNA containing the Aspergillus nidulans amdS gene with its native promoter and terminator regions.

TABLE 2
Chlorophyllases from different plants and
database accession numbers.

| Source of Chlorophyllase Genes | Accession No. |
|---|---|
| Arabidopsis thaliana At2CHL | Q9M7I7 |
| Brassica oleracea | Q8GTM3 |
| Ricinus communis | XP_002517075 |
| Populus trichocarpa | B9HUR3 |
| Ginkgo biloba | Q7Y0K5 |
| Vitis vinifera | XP002273926 |
| Phyllostachys heterocycla | FP092915 |
| Sorghum bicolor | C5YN66 |
| Glycine max | BAF43704 |
| Pachira macrocarpa | C1JZ62 |
| Triticum aestivum | BT009214 |
| Brassica oleracea | Q8GTM4 |
| Sorghum bicolour | C5X5Z9 |
| Citrus sinensis | Q9MV14 |
| Zea mays (Maize) | C0PCYS |
| Chenopodium album | Q9LE89 |
| Picea sitchensis | ACN40275 |

Secreted Versions of Chlorophyllases from Wheat and Algae.

Constructs for secretion of chlorophyllases from wheat and chlamydomonas were generated by fusing 10 different signal peptide sequences, derived from known secreted proteins of the Trichoderma secretome, in front of the chlorophyllase gene for extracellular deposition/secretion of chlorophyllases. The following signal peptide-encoding sequences were used, as shown in Table 3:

TABLE 3

| Source of | |
|---|---|
| signal | |
| sequence | Signal peptide-encoding sequence fused to wheat chlorophyllase gene |
| 1, Cbh2 | caccatgattgtcggcattctcaccacgctggctacgctggccacactcgcagctagtgtgcctctagagg |
| | agcggactagtgcg (SEQ ID NO: 39) |
| 2. Cbh1 | caccatgtatcggaagttggccgtcatctcggccttcttggccacagctcgtgctcagtcgactagt |
| | (SEQ ID NO: 40) |
| 3. lip3 | caccatgttactggacggtttggagtgcttttgacagcgcttgctgcgctgggtgctgccgcgccggcacc |
| prepro | gcttgctgtgcggagtaggtgtgcccgatgtgagatggttggatagcactgatgaagggtgaataggtgtc |
| | tcgactagt (SEQ ID NO: 41) |
| 4. TrGa | caccatgcacgtcctgtcgactgcggtgctgctcggctccgttgccgttcaaaaggtcctgggaagaccaa |
| | ctagt (SEQ ID NO: 42) |
| 5. cbh2 | caccatgattgtcggcattctcaccacgctggctacgctggccacactcgcagctagtgtgcctctagagg |
| linker | agcggcaagcttgctcaagcgtctggtaattatgtgaaccactcaagagacccaaatactgagatatgtca |
| | aggggccaatgtggtggccagaattggtcgggtccgacttgctgtgcttccggaagcacatgcgtctactc |
| | caacgactattactcccagtgtcttcccggcgctgcaagctcaagctcgtccacgcgcgccgcgtcgacg |
| | acttctcgagtatcccccacaacatcccggtcgagctccgcgacgcctccacctggttctactactaccaga |
| | gtacctccagtcggaactagt (SEQ ID NO: 43) |
| 6. eg2ss | caccatgaacaagtccgtggctccattgctgcttgcagcgtccatactatatggcggcgccgctgcacagc |
| linker | agactgtctggggccagtgtggaggtattggttggagcggacctacgaattgtgctcctggctcagatgtt |
| | cgaccacaatccttattatgcgcaatgattccgggagccactactatcaccacttcgacccggccaccat |
| | ccggtccaaccaccaccaccagggctacctcaacaagctcatcaactccacccacgagctctgggacta |
| | gt (SEQ ID NO: 44) |
| 7. eg1 | caccatggcgccctcagttacactgccgttgaccacggccatcctggccattgcccggctcgtcgccgcc |
| | actagt (SEQ ID NO: 45) |
| 8. eg6 | caccatgaaggtctctcgagtccttgcccttgtcctgggggccgtcatccctgcccatgctgcctttactagt |
| | (SEQ ID NO: 46) |
| 9. xyn1 | caccatggttgccttttccagcctcatctgcgctctcaccagcatcgccagtactctggcgatgcccactagt |
| | (SEQ ID NO: 47) |
| 10. xyn3 | caccatgaaagcaaacgtcatcttgtgcctcctggcccccctggtcgccgctctccccactagt |
| | (SEQ ID NO: 48) |

Transformation of Fungi with the Chlorophyllase Gene Constructs

Introduction of the chlorophyllase gene constructs into a production host such as yeasts (Pichia pastoris, Hansenula polymorpha) and filamentous fungi (Aspergillus and Trichoderma) are carried out either by electroporation or biolistic using spores or by making protoplasts.

Expression vectors separately containing each of the chlorophyllase genes of interest were transformed into a T. reesei host strain derived from RL-P37 (1A52) and having 4 gene deletions (cbh1, cbh2, eg1, eg2) using biolistic transformation (particle bombardment using the PDS-1000 Helium system, BioRad Cat. No 165-02257) methods. Transformation by biolistic transformation was performed as follows:

A suspension of spores (approximately 5×10⁸ spores/ml) from the T. reesei host strain was prepared. 100-200 μL of spore suspension was spread onto the center of plates containing minimal medium acetamide. The spore suspension was allowed to dry on the surface of the plates. Transformation followed the manufacturer's protocol. Briefly, 1 mL ethanol was added to 60 mg of M10 tungsten particles in a microcentrifuge tube and the suspension was allowed to stand for 15 seconds. The particles were centrifuged at 15,000 rpm for 15 seconds. The ethanol was removed and the particles were washed three times with sterile H2O before 1 mL of 50% (v/v) sterile glycerol was added to them. 25 μL of tungsten particle suspension was placed into a microtrifuge tube. While continuously vortexing, the following were added: 5 μL (100-200 ng/μL) of plasmid DNA, 25 μL of 2.5M CaCl2 and 10 μL of 0.1 M spermidine. The particles were centrifuged for 3 seconds. The supernatant was removed and the particles were washed with 200 μL of 100% ethanol and centrifuged for 3 seconds. The supernatant was removed and 24 μL of 100% ethanol was added to the particles and mixed. Aliquots of 8 μL of particles were removed and placed onto the center of macrocarrier disks that were held in a desiccator. Once the tungsten/DNA solution had dried the macrocarrier disk was placed in the bombardment chamber along with the plate of minimal medium acetamide with spores and the bombardment process was performed according to the manufacturer's protocol. After bombardment of the plated spores with the tungsten/DNA particles, the plates were incubated at 30° C.

### Chlorophyllase Expression and Screening

Transformants were picked and transferred individually to acetamide agar plates. After 5 days of growth on minimal medium acetamide plates, transformants displaying stable morphology were inoculated into 10 ml YEG media, grown for 2 days with shaking at 28° C. After 2 days of growth, 5 mls culture is used to inoculate a 250 ml flask containing 50 mls Glucose/Sophorose defined media. Glucose/Sophorose defined medium (per liter) consists of (NH4)2SO4, 5 g; PIPPS buffer, 33 g; Casamino Acids, 9 g; KH2PO4, 4.5 g; CaCl2 (anhydrous), 1 g, MgSO4.7H2O, 1 g; pH 5.50 adjusted with 50% NaOH with sufficient milli-Q H2O to bring to 966.5 mL. After sterilization, the following were added: 5 mL Mazu, 26 mL 60% Glucose/Sophrose, and 400× T. reesei Trace Metals 2.5 mL.

The cultures were incubated with shaking at 28° C. for 4 days. Cells and supernatants from these cultures were collected by centrifugation. Cells were lyzed and chlorophyllase activity assayed in cellular protein extracts or in culture supernatants. The protein extracts were characterized by SDS-Page electrophoresis and chlorophyllase specific protein bands identified by western blots. A wheat chlorophyllase antibody was used to screen transformants producing the different chlorophyllases. The antibody crossreacts with all the different plant chlorophyllases tested.

### Large Scale Production of Recombinant Chlorophyllase

For large scale protein production, T. reesei transformants were cultured in fey nenters as described in WO 2004/035070. Ultrafiltered concentrate (UFC) from tanks or ammonium sulfate purified protein samples were used for biochemical assays.

In particular, Trichoderma transformant I-7 harbouring the wheat chlorophyllase expression construct was grown in standard defined Trichoderma media. The transformant was pre-grown in a flask with shaking at 34° C. and pH 3.5 until glucose is depleted. Then glucose/sophrose feed is started and temperature is shifted from 34 to 28 C as well as pH from 3.5 to 4. Glucose/sophrose is used as the inducer of the cbh1 promoter. DO % is kept constant by adjusting agitation, pressure and airflow. The runs go for 200 hours dependent on the rate of production.

### Chlorophyllase Assay

The activities of the expressed recombinant chlorophyllases were determined using pheophytin as the substrate. The assay may be performed as described in EP10159327.5. The assay relies on the fact that pheophytin in the dilution buffer forms a dimer, which quenches the fluorescence signal of pheophytin. The dilution buffer is formulated such that pheophorbide produced does not form a dimer and thus can be detected by fluorescence spectroscopy. The activity unit PHEU (Pheophytinase Units) is defined according to a standard enzyme.

### Results

Table 4 below shows chlorophyllase activity measurements of recombinant strains harbouring the wheat chlorophyllase intracellular gene construct (SMMI1-SMMI12), and transformants containing the chlorophyllase gene fused to signal peptides (SMMT2, T4 and T6).

TABLE 4

| | | | | | Activity |
|---|---|---|---|---|---|
| | | Average | sample- | μM | μmol/min |
| | F | RFU | Blank | Pheophorbide | per ml |
| blank | 1 | 35.389 | | | |
| Arabidopsis | 50 | 219.585 | 184.196 | 0.095364 | 0.13 |
| chlorophyllase | | | | | |
| SMM I1 | 200 | 336.3 | 300.911 | 0.155791 | 0.87 |
| SMM I2 | 200 | 779.1965 | 743.8075 | 0.385093 | 2.16 |
| SMM I3 | 200 | 809.8495 | 774.4605 | 0.400963 | 2.25 |
| SMM I4 | 200 | 354.581 | 319.192 | 0.165256 | 0.93 |
| SMM I5 | 200 | 768.565 | 733.176 | 0.379589 | 2.13 |
| SMM I6 | 200 | 34.075 | −1.314 | −0.00068 | 0.00 |
| SMM I7 | 200 | 938.5375 | 903.1485 | 0.467589 | 2.62 |
| SMM I8 | 200 | 513.3825 | 477.9935 | 0.247473 | 1.39 |
| blank | 1 | 37.538 | 2.149 | 0.001113 | 0.00 |
| SMM I9 | 200 | 487.004 | 451.615 | 0.233816 | 1.31 |
| SMM I10 | 200 | 485.7275 | 450.3385 | 0.233155 | 1.31 |
| SMM I11 | 200 | 229.695 | 194.306 | 0.100598 | 0.56 |
| SMM I12 | 200 | 232.216 | 196.827 | 0.101904 | 0.57 |
| negative | 1 | 45.8605 | 10.4715 | 0.005421 | 0.00 |
| control | | | | | |
| SMM I2 | 400 | 521.826 | 486.437 | 0.251844 | 2.82 |
| SMM I3 | 400 | 570.958 | 535.569 | 0.277281 | 3.11 |
| SMM I5 | 400 | 500.3685 | 464.9795 | 0.240735 | 2.70 |
| SMM I7 | 400 | 748.7275 | 713.3385 | 0.369318 | 4.14 |
| blank | | 37.5015 | | 0 | 0.00 |
| +ve control | 9000 | 218.368 | 180.8665 | 0.09364 | 23.60 |
| chlorophyllase | | | | | |
| SMM I2 | 1000 | 239.6855 | 202.184 | 0.104677 | 2.93 |
| SMM I3 | 1000 | 241.275 | 203.7735 | 0.1055 | 2.95 |
| SMM I5 | 1000 | 213.2285 | 175.727 | 0.09098 | 2.55 |
| SMM I7 | 1000 | 289.382 | 251.8805 | 0.130407 | 3.65 |
| SMM I8 | 1000 | 156.6955 | 119.194 | 0.061711 | 1.73 |
| SMM I9 | 400 | 213.925 | 176.4235 | 0.09134 | 1.02 |
| SMM I10 | 400 | 202.584 | 165.0825 | 0.085469 | 0.96 |
| T2-1 | 10 | 128.162 | 90.6605 | 0.046938 | 0.01 |
| blank | | 43.702 | | 0 | 0.00 |
| +ve control | 9000 | 239.8035 | 196.1015 | 0.101528 | 25.59 |
| chlorophyllase | | | | | |
| SMM T2-5 | 10 | 225.1555 | 181.4535 | 0.093944 | 0.03 |
| SMM T2-6 | 10 | 152.6535 | 108.9515 | 0.056408 | 0.02 |
| SMM T2-11 | 10 | 133.4025 | 89.7005 | 0.046441 | 0.01 |
| SMM T2-12 | 10 | 108.3615 | 64.6595 | 0.033476 | 0.01 |
| SMM T2-13 | 10 | 240.3215 | 196.6195 | 0.101796 | 0.03 |
| SMM T4-1 | 10 | 55.947 | 12.245 | 0.00634 | 0.00 |
| SMM T4-2 | 10 | 57.1865 | 13.4845 | 0.006981 | 0.00 |
| SMM T4-4 | 10 | 71.7875 | 28.0855 | 0.014541 | 0.00 |
| blank | | 36.789 | | 0 | 0.00 |
| +ve control | 9000 | 228.2545 | 191.4655 | 0.099128 | 24.98 |
| chlorophyllase | | | | | |
| SMM T6-4 | 10 | 76.152 | 39.363 | 0.020379 | 0.01 |
| SMM T6-5 | 10 | 91.599 | 54.81 | 0.028377 | 0.01 |
| SMM T6-10 | 10 | 146.904 | 110.115 | 0.05701 | 0.02 |

The transformed Trichoderma strain expressing the highest amount of recombinant wheat chlorophyllase in shake flask cultures was subjected to large scale fermentation in 14 litre scale for 150 hours. This strain harboured a wheat chlorophyllase intracellular gene construct. Whole cell broth was centrifuged and both intracellular and extracellular protein fractions from the cell pellet and supernatant respectively were analyzed by SDS-PAGE.

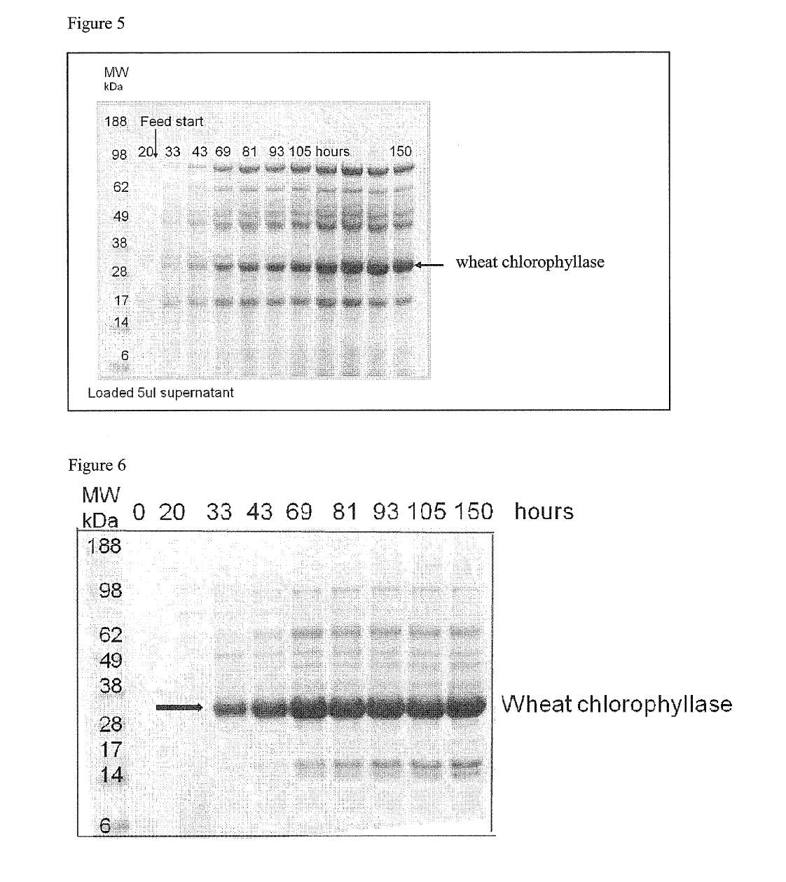

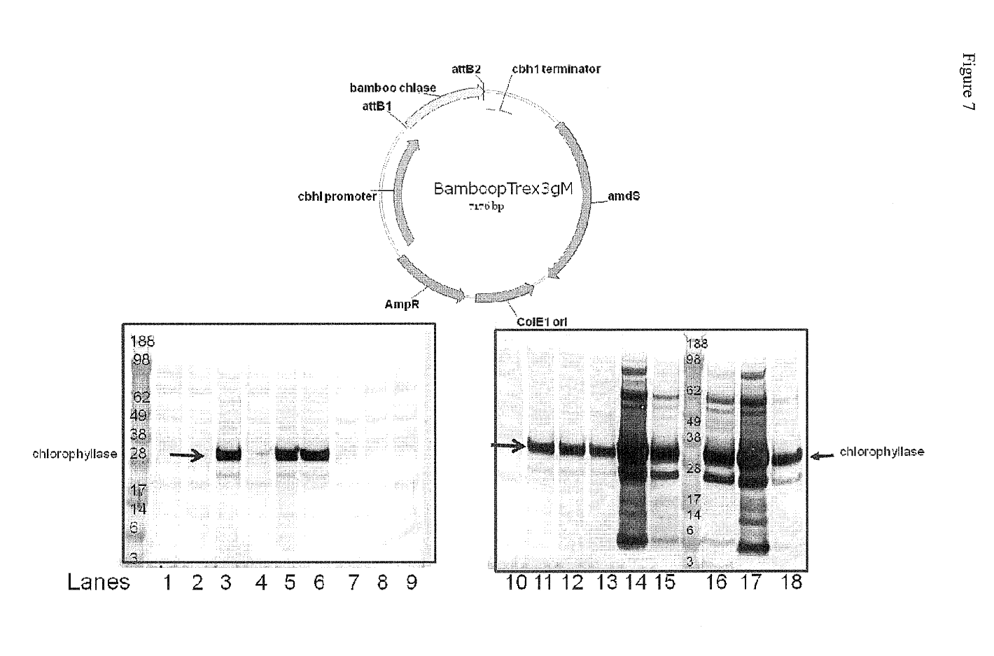

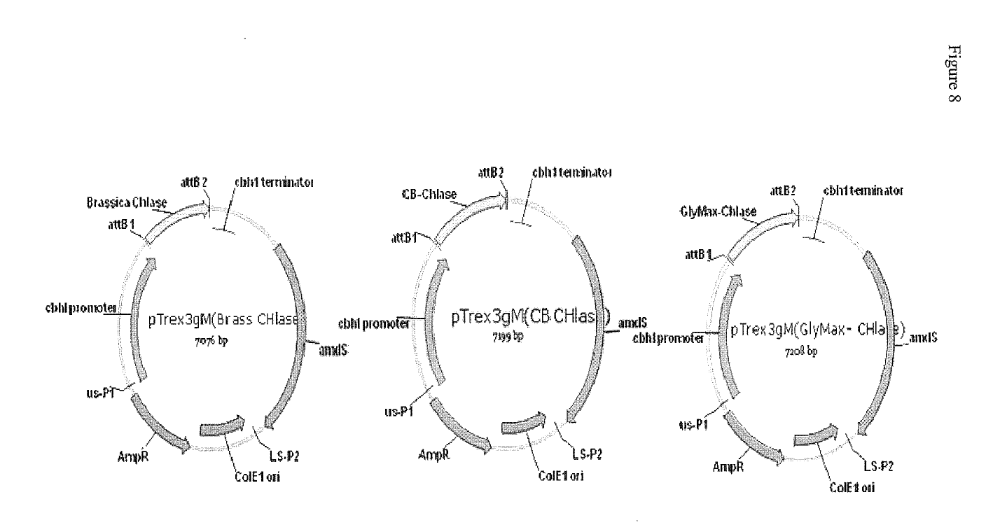

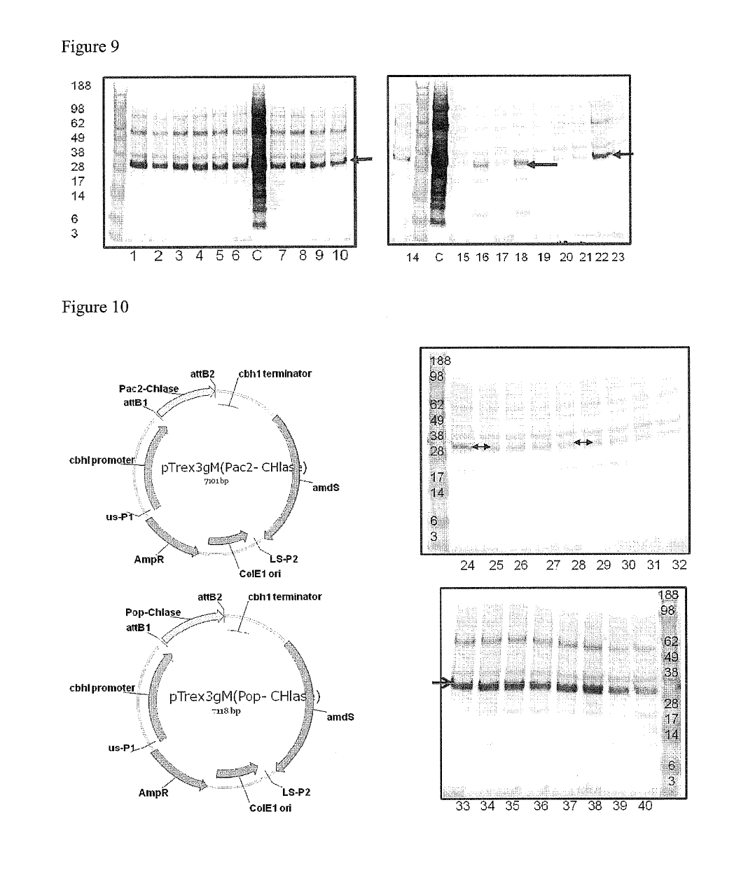

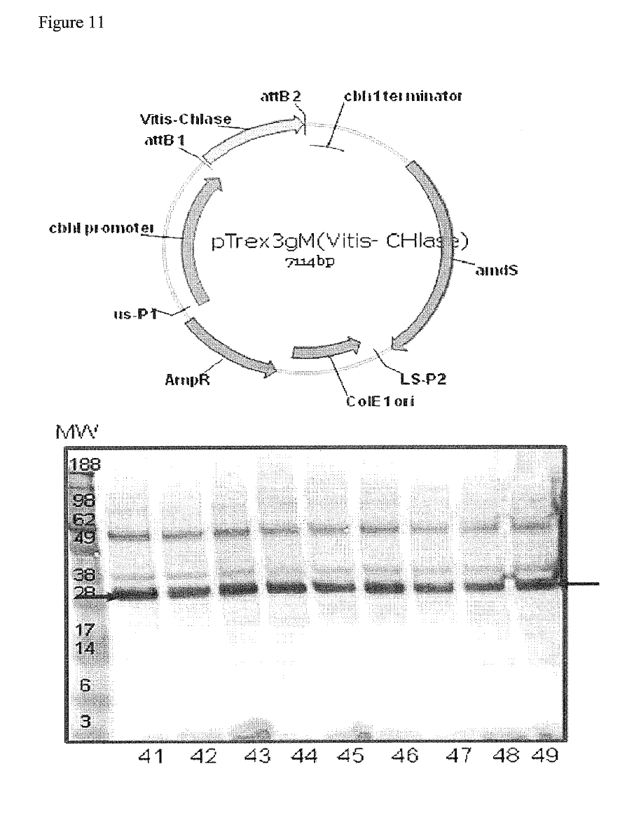

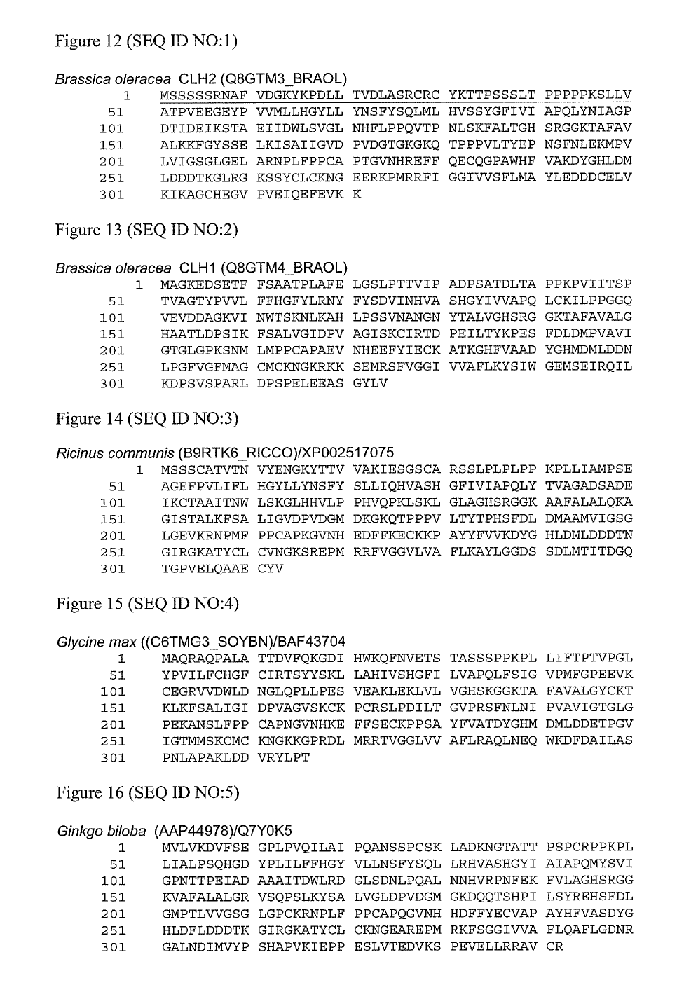

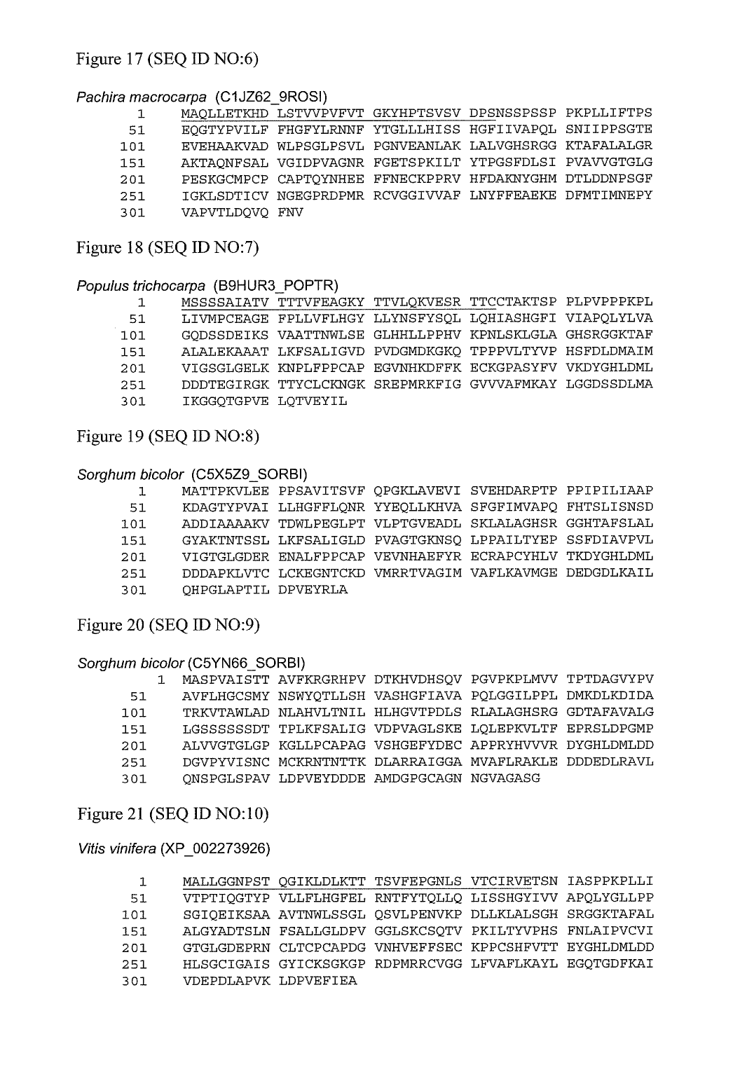

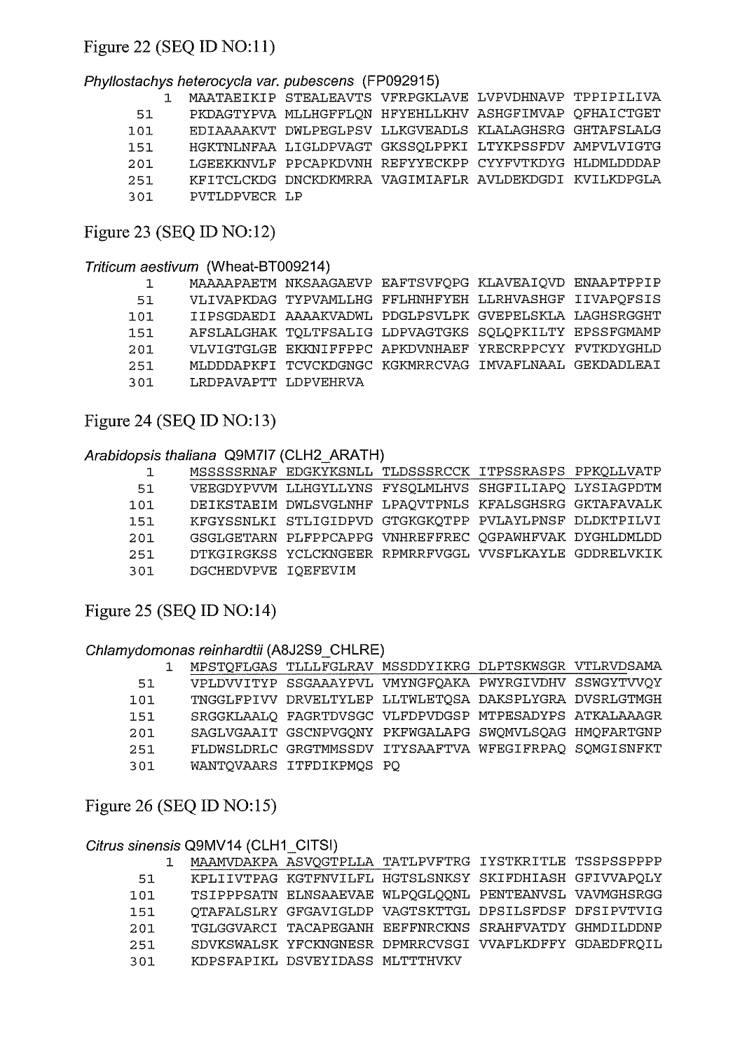

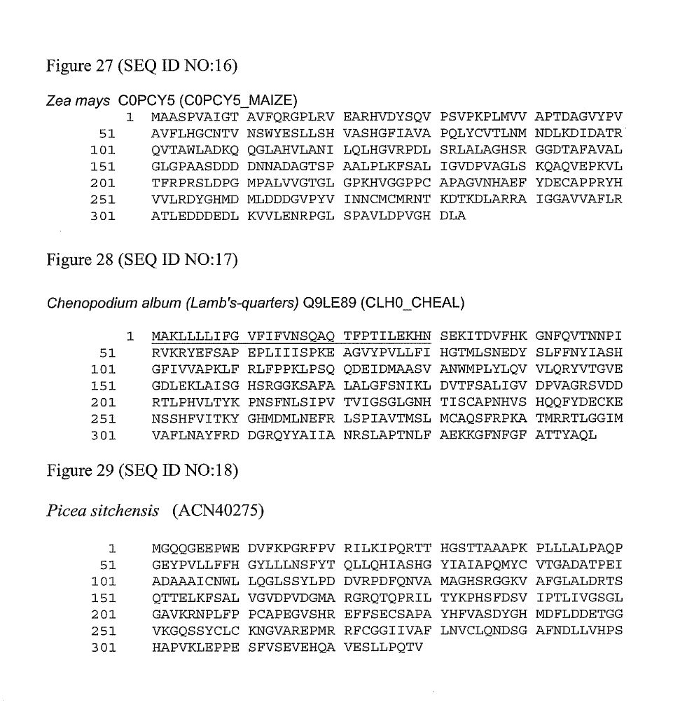

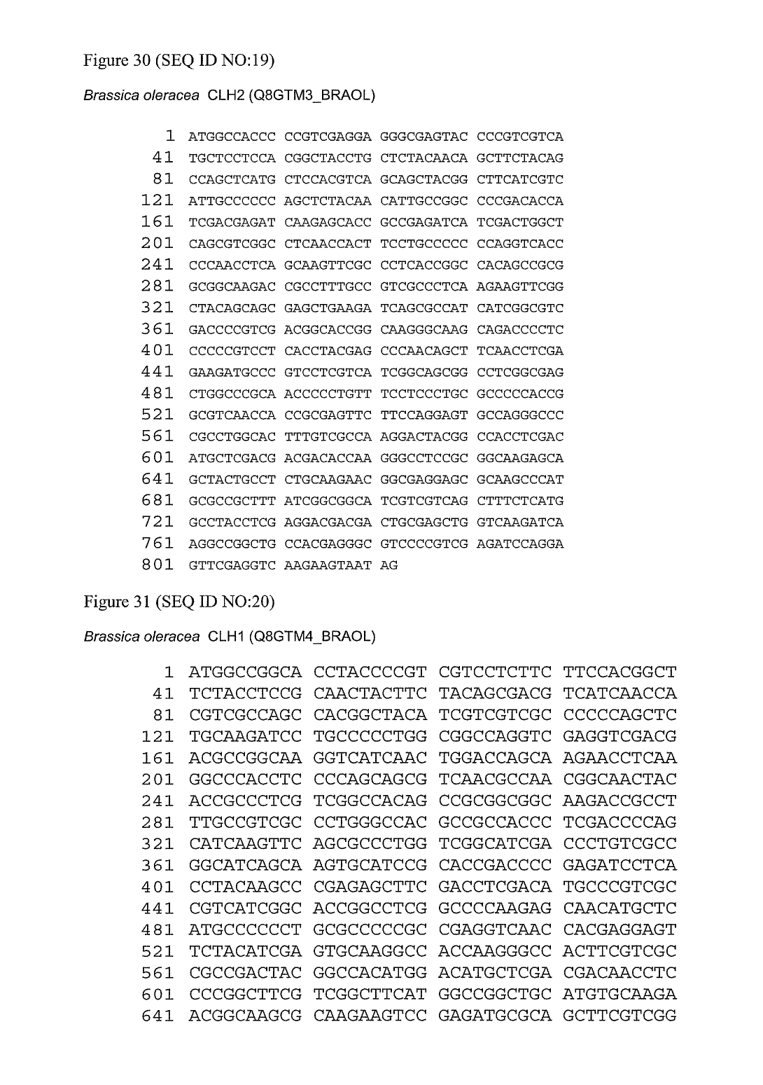

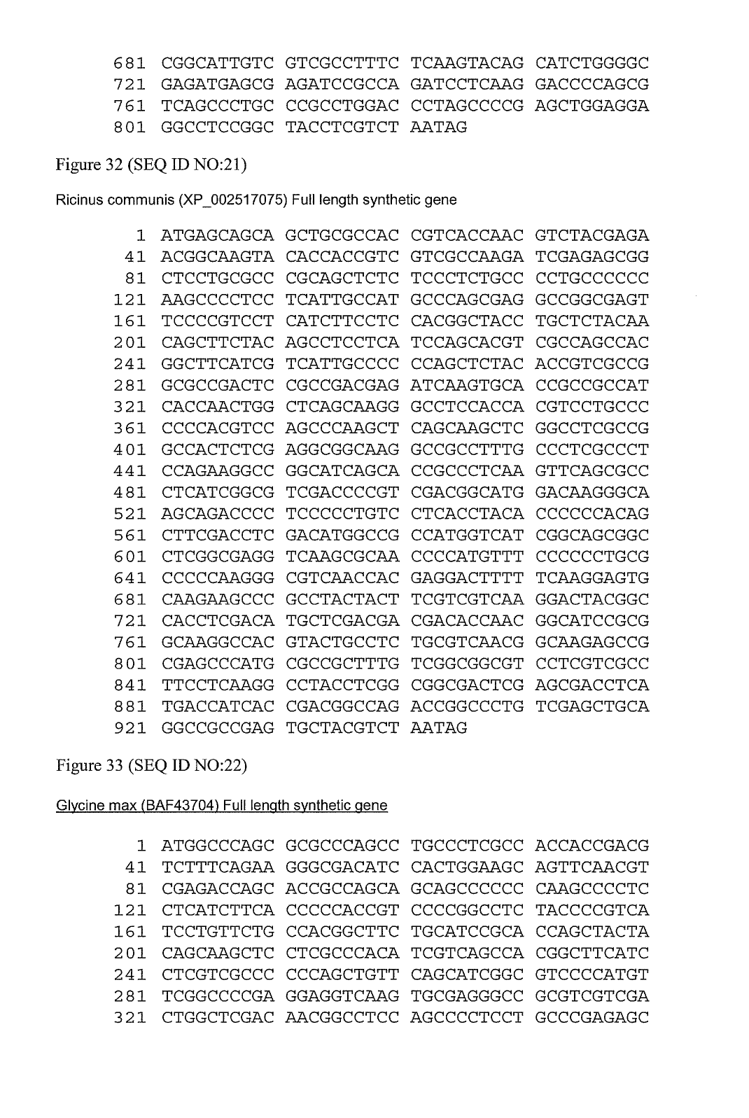

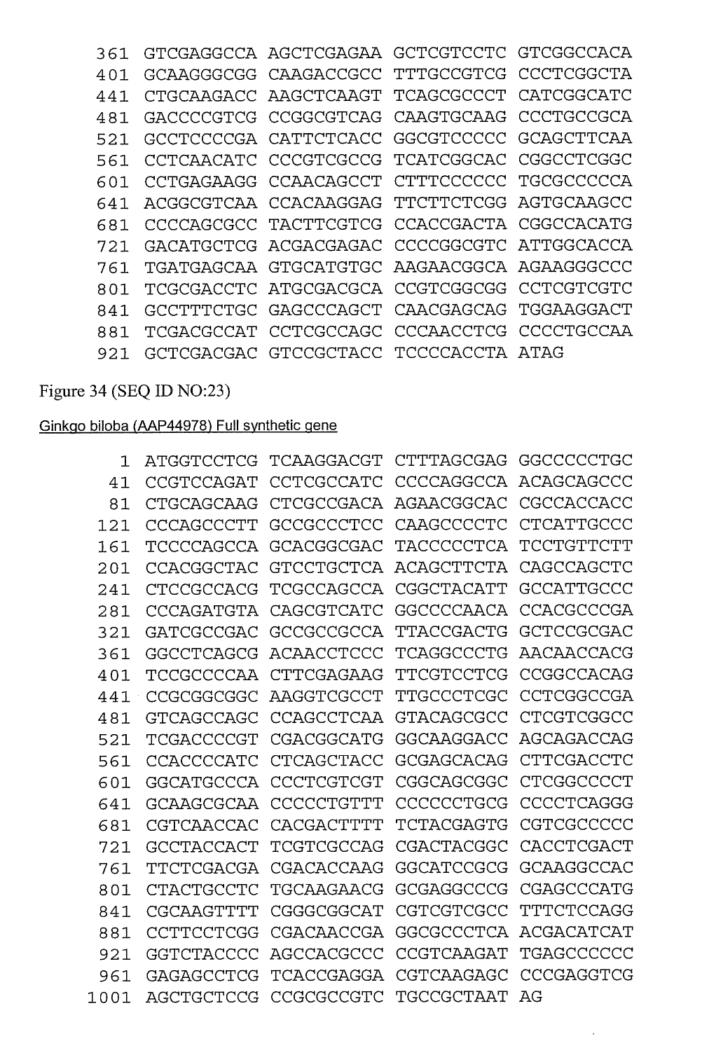

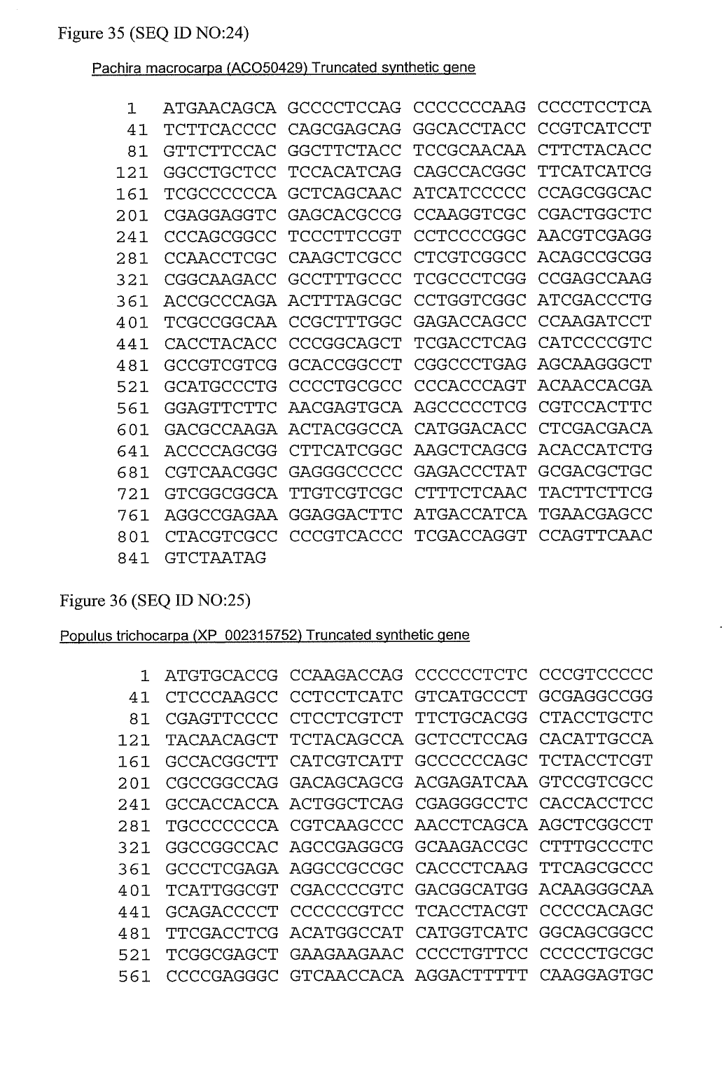

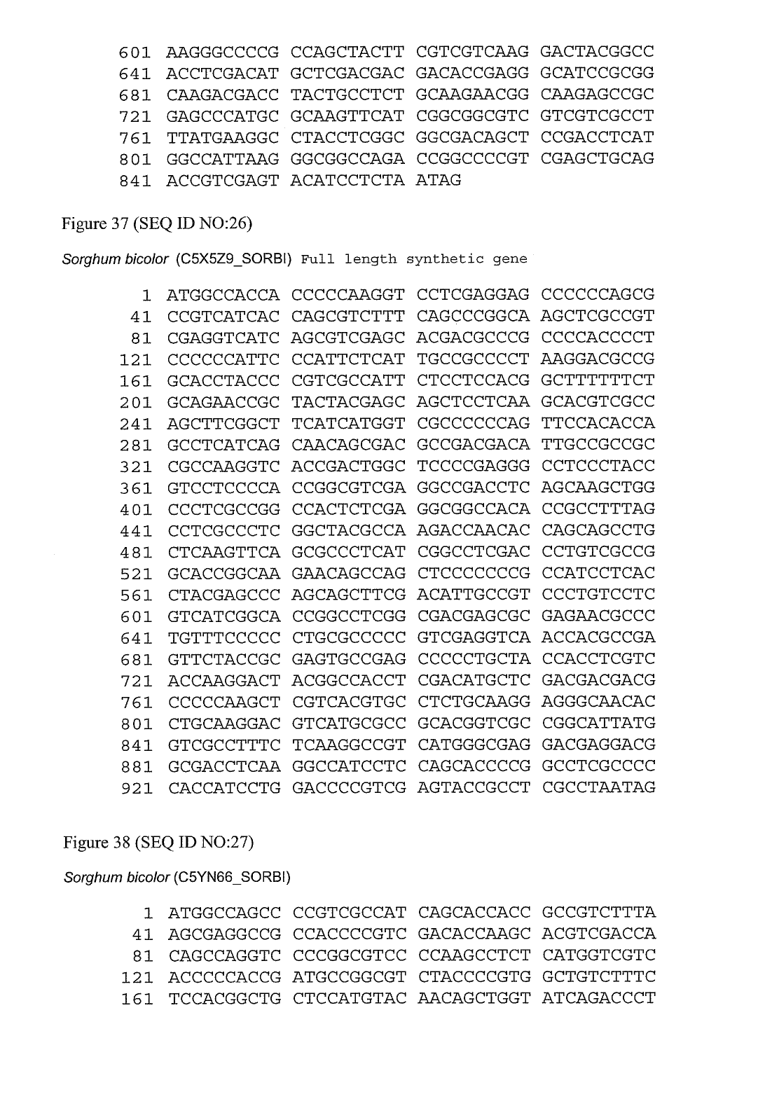

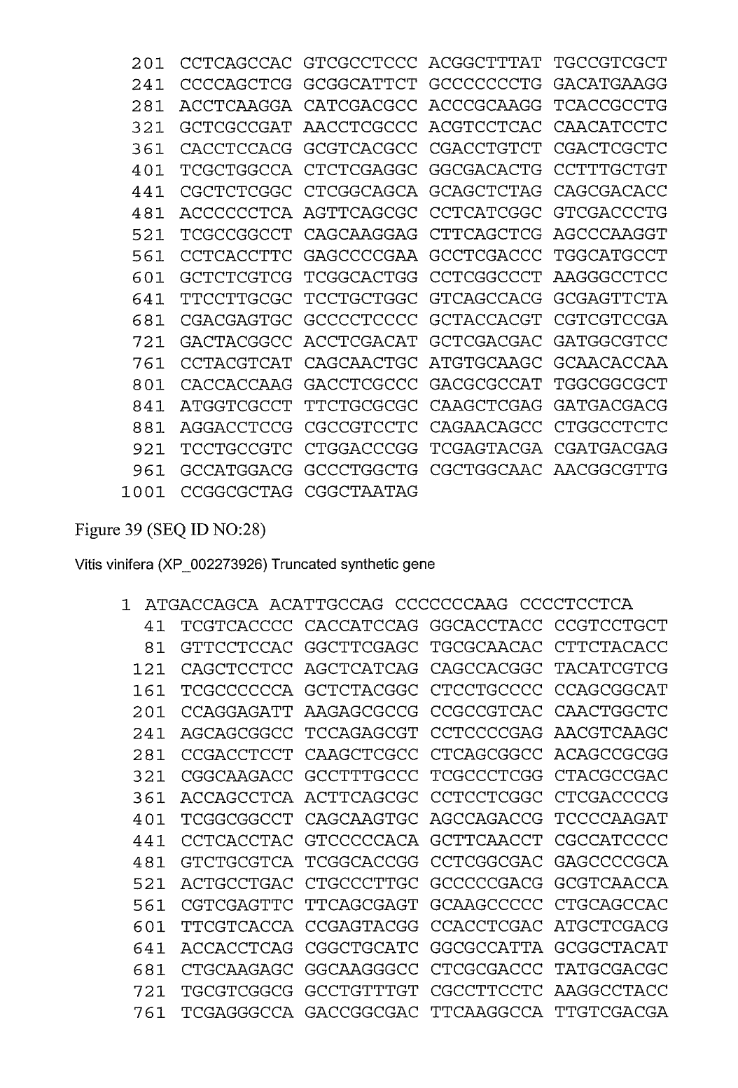

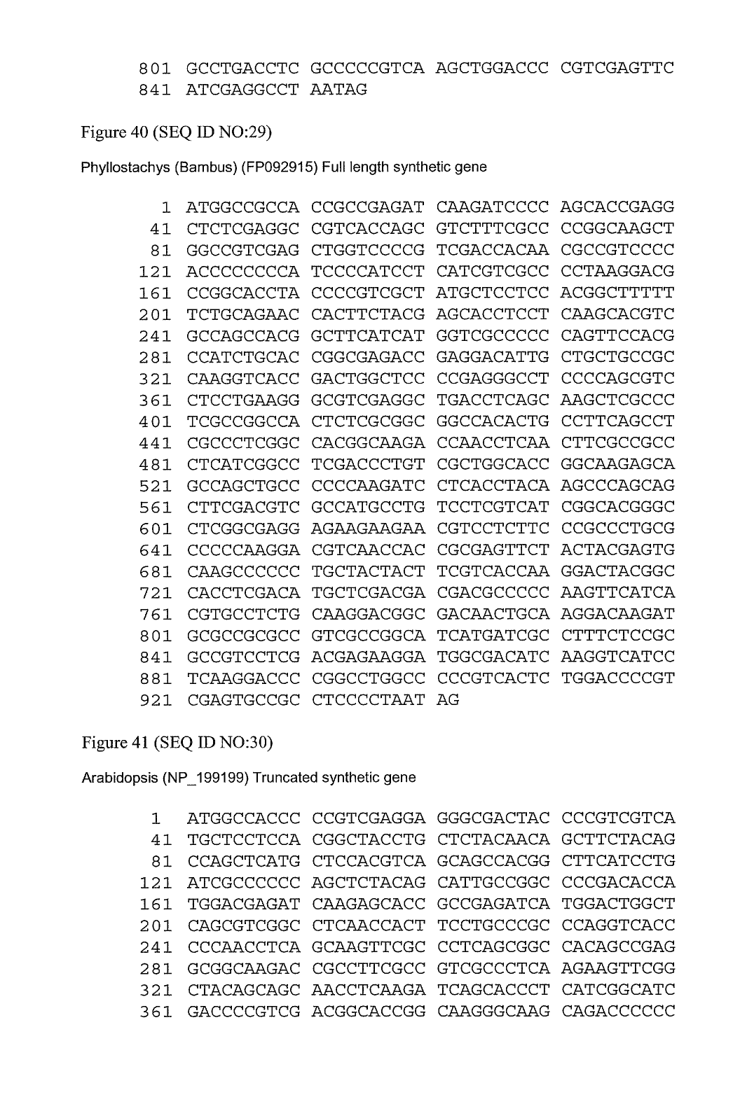

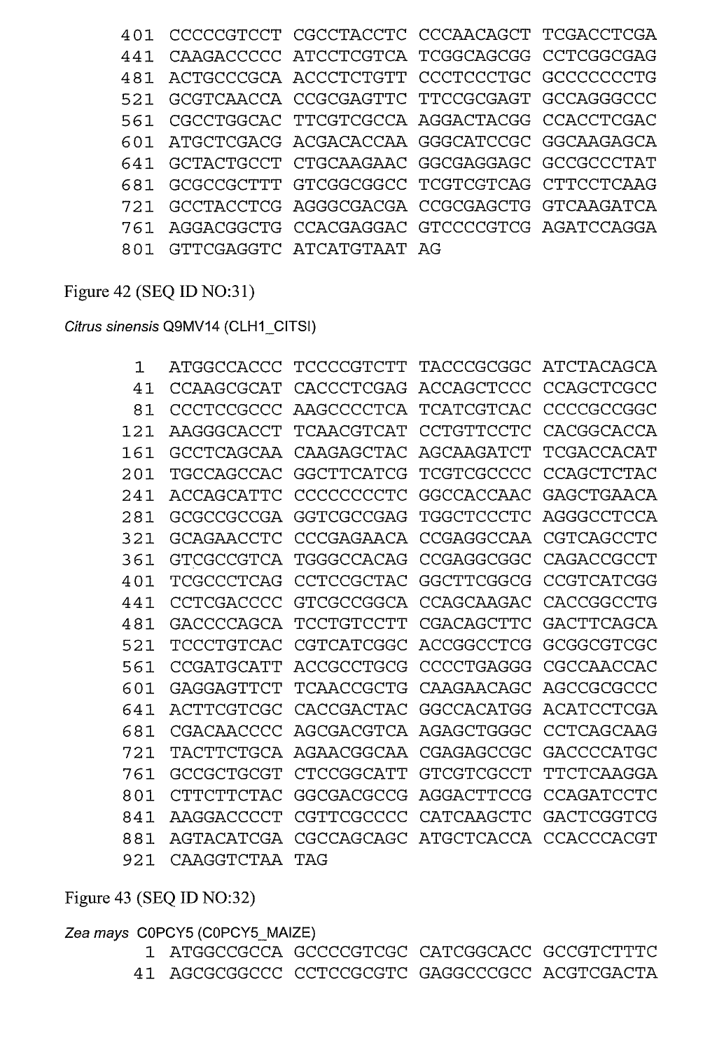

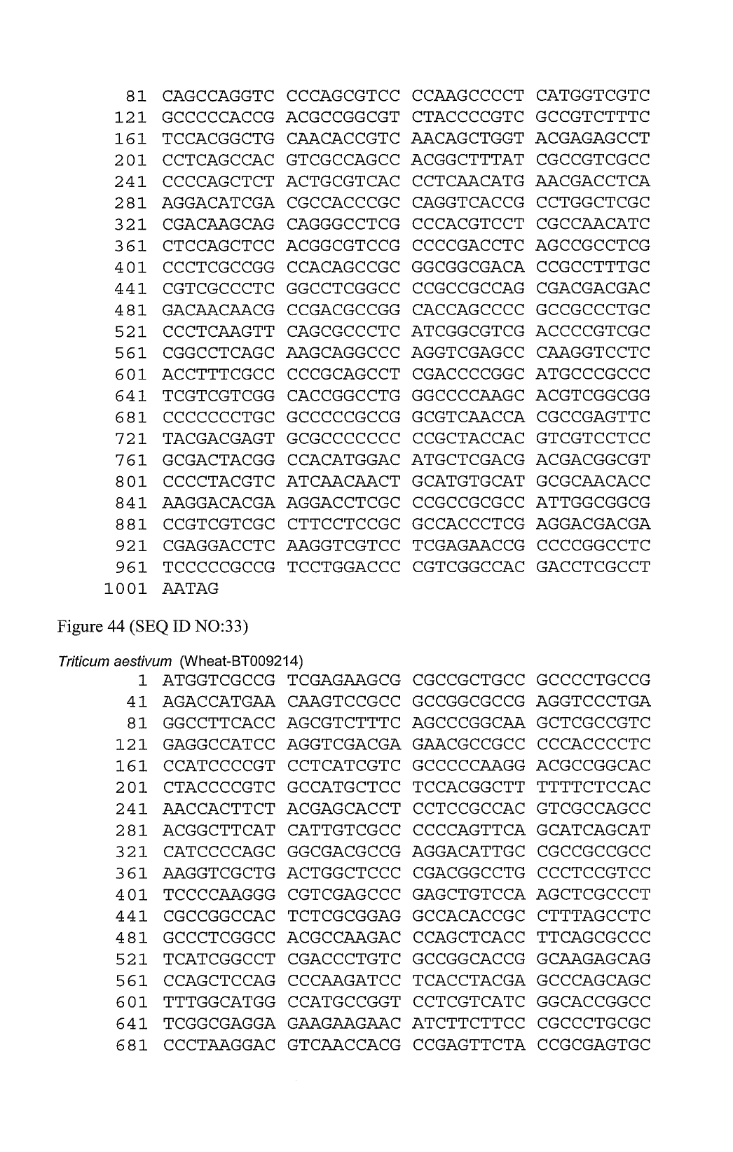

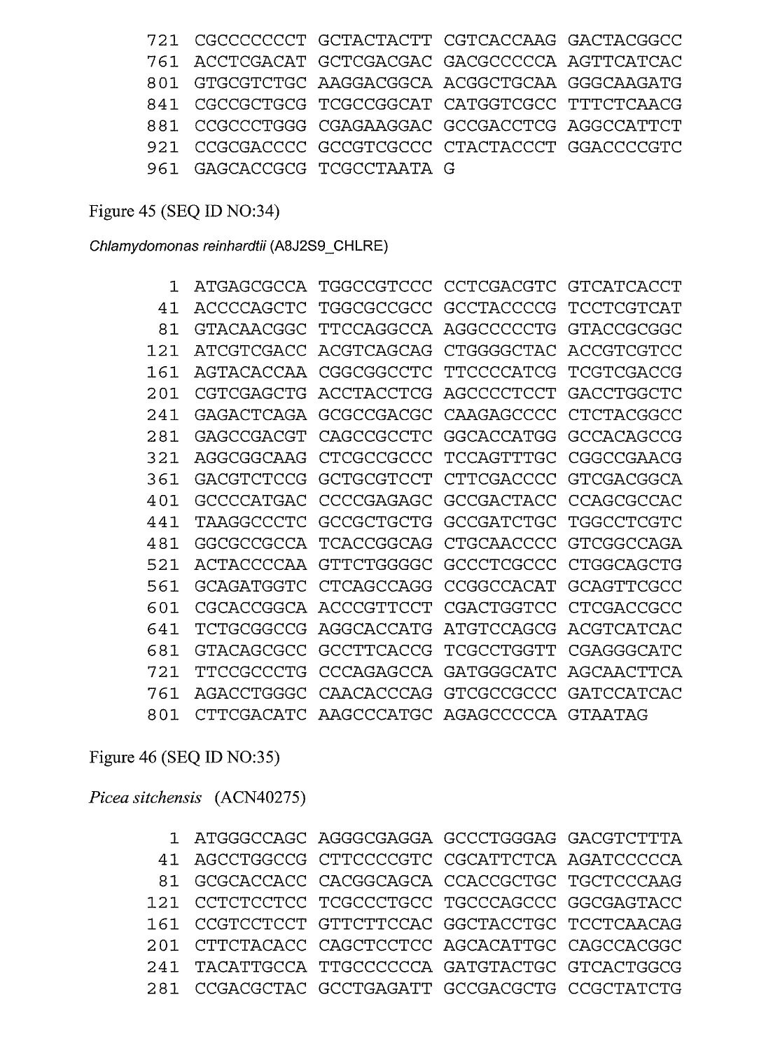

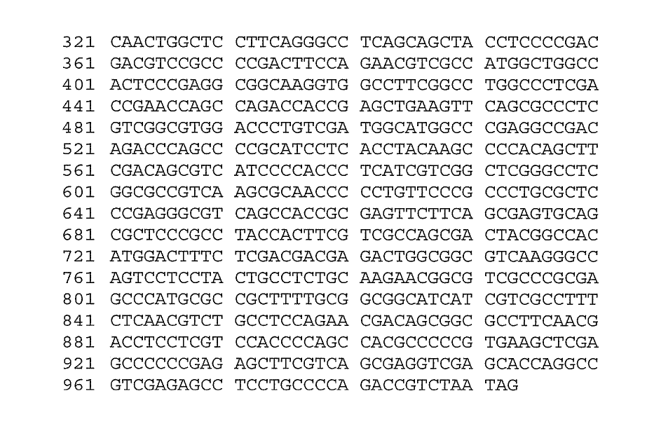

Cell free extracts of culture supernatant from 14 litre scale fermentation showed the presence of recombinant wheat chlorophyllase both extracellularly and intracellularly. Wheat chlorophyllase was the most abundant protein accumulating outside of fungal cells (see FIG. 5). The extracellular accumulation of chlorophyllase in the culture medium could be explained by the following possibilities: 1. chlorophyllase proteins can cross plasma membranes and cell walls, 2. The occurrence of cell lysis with age of the mycelia being more prone to cell breakage. The cell pellet was lysed using celLytic buffer to check the intracellular protein fraction for the accumulation of chlorophyllase intracellularly. As shown in FIG. 6, SDS-PAGE indicated the presence of a 34 kDa wheat chlorophyllase as the predominant protein expressed inside the fungal cells.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A method for producing a recombinant enzyme capable of hydrolysing chlorophyll or a chlorophyll derivative, comprising a step of intracellular expression of the recombinant enzyme in a host cell, wherein the enzyme is not targeted for extracellular secretion.

2. (canceled)

3. A method according to claim 1, wherein the host cell is derived from Trichoderma spp.

4. A method according to claim 1, wherein the recombinant enzyme is a chlorophyllase.

5. A method according to claim 1, wherein the enzyme comprises one or more amino acid sequences selected from GHSXGG (SEQ ID NO:36), DPVXG (SEQ ID NO:37) and YGHXD (SEQ ID NO:38).

6. A method according to claim 1, wherein the recombinant enzyme is derived from Arabidopsis thaliana, Brassica oleracea, Ricinus communis, Ginkgo biloba, Populus trichocarpa, Vitis vinifera, Phyllostachys heterocycle, Sorghum bicolor, Glycine max, Pachira macrocarpa, Triticum aestivum, Citrus sinensis, Zea mays, Chenopodium album, Picea sitchensis or Chlamydomonas reinhardtii.

7. method according to claim 1, wherein the recombinant enzyme comprises a polypeptide sequence as defined in any one of SEQ ID NOs: 1 to 18, or a functional fragment or variant thereof having at least 75% sequence identity to any one of SEQ ID Nos: 1 to 18 over at least 50 amino acid residues.

8. A method according to claim 1, wherein the recombinant enzyme is encoded by a nucleic acid sequence as defined in any one of SEQ ID Nos: 19 to 35, or a functional fragment or variant thereof having at least 75% sequence identity to any of SEQ ID NOs: 19 to 35 over at least 50 nucleotide residues.

9. A method according to claim 1, further comprising lysing the host cells and recovering the recombinant enzyme from the lysate.

10. A method according to claim 9, wherein the host cells are exposed to a detergent, an organic acid, homogenization and/or a heat treatment.

11. A eukaryotic expression vector, comprising a nucleic acid sequence encoding an enzyme capable of hydrolysing chlorophyll or a chlorophyll derivative, operably linked to one or more control sequences suitable for directing intracellular expression of the enzyme in a fungal host cell.

12. An expression vector according to claim 11, comprising a nucleic acid sequence as defined in any one of SEQ ID NOs: 19 to 35, or a functional fragment or variant thereof having at least 75% sequence identity to any of SEQ ID NOs: 19 to 35 over at least 50 nucleotide residues.

13. A fungal host cell comprising an expression vector as defined in claim 11.

14. (canceled)

15. A recombinant enzyme capable of hydrolyzing chlorophyll or a chlorophyll derivative, which is obtainable by a method as defined in claim 1.

16. A recombinant enzyme according to claim 15, wherein the recombinant enzyme comprises a polypeptide sequence as defined in any one of SEQ ID NOs: 1 to 18, or a functional fragment or variant thereof having at least 75% sequence identity to any one of SEQ ID NOs: 1 to 18 over at least 50 amino acid residues.


SEQUENCE LISTING (73 sequences)

SEQ ID NO: 1 - protein, 321 aa, Brassica oleracea
MSSSSSRNAFVDGKYKPDLLTVDLASRCRCYKTTPSSSLTPPPPPKSLLVATPVEEGEYPVVMLLHGYLLYNSFYSQLMLHVSSYGFIVIAPQLYNIAGPDTIDEIKSTAEIIDWLSVGLNHFLPPQVTPNLSKFALTGHSRGGKTAFAVALKKFGYSSELKISAIIGVDPVDGTGKGKQTPPPVLTYEPNSFNLEKMPVLVIGSGLGELARNPLFPPCAPTGVNHREFFQECQGPAWHFVAKDYGHLDMLDDDTKGLRGKSSYCLCKNGEERKPMRRFIGGIVVSFLMAYLEDDDCELVKIKAGCHEGVPVEIQEFEVKK

SEQ ID NO: 2 - protein, 324 aa, Brassica oleracea
MAGKEDSETFFSAATPLAFELGSLPTTVIPADPSATDLTAPPKPVIITSPTVAGTYPVVLFFHGFYLRNYFYSDVINHVASHGYIVVAPQLCKILPPGGQVEVDDAGKVINWTSKNLKAHLPSSVNANGNYTALVGHSRGGKTAFAVALGHAATLDPSIKFSALVGIDPVAGISKCIRTDPEILTYKPESFDLDMPVAVIGTGLGPKSNMLMPPCAPAEVNHEEFYIECKATKGHFVAADYGHMDMLDDNLPGFVGFMAGCMCKNGKRKKSEMRSFVGGIVVAFLKYSIWGEMSEIRQILKDPSVSPARLDPSPELEEASGYLV

SEQ ID NO: 3 - protein, 313 aa, Ricinus communis
MSSSCATVTNVYENGKYTTVVAKIESGSCARSSLPLPLPPKPLLIAMPSEAGEFPVLIFLHGYLLYNSFYSLLIQHVASHGFIVIAPQLYTVAGADSADEIKCTAAITNWLSKGLHHVLPPHVQPKLSKLGLAGHSRGGKAAFALALQKAGISTALKFSALIGVDPVDGMDKGKQTPPPVLTYTPHSFDLDMAAMVIGSGLGEVKRNPMFPPCAPKGVNHEDFFKECKKPAYYFVVKDYGHLDMLDDDTNGIRGKATYCLCVNGKSREPMRRFVGGVLVAFLKAYLGGDSSDLMTITDGQTGPVELQAAECYV

SEQ ID NO: 4 - protein, 316 aa, Glycine max
MAQRAQPALATTDVFQKGDIHWKQFNVETSTASSSPPKPLLIFTPTVPGLYPVILFCHGFCIRTSYYSKLLAHIVSHGFILVAPQLFSIGVPMFGPEEVKCEGRVVDWLDNGLQPLLPESVEAKLEKLVLVGHSKGGKTAFAVALGYCKTKLKFSALIGIDPVAGVSKCKPCRSLPDILTGVPRSFNLNIPVAVIGTGLGPEKANSLFPPCAPNGVNHKEFFSECKPPSAYFVATDYGHMDMLDDETPGVIGTMMSKCMCKNGKKGPRDLMRRTVGGLVVAFLRAQLNEQWKDFDAILASPNLAPAKLDDVRYLPT

SEQ ID NO: 5 - protein, 342 aa, Ginkgo biloba
MVLVKDVFSEGPLPVQILAIPQANSSPCSKLADKNGTATTPSPCRPPKPLLIALPSQHGDYPLILFFHGYVLLNSFYSQLLRHVASHGYIAIAPQMYSVIGPNTTPEIADAAAITDWLRDGLSDNLPQALNNHVRPNFEKFVLAGHSRGGKVAFALALGRVSQPSLKYSALVGLDPVDGMGKDQQTSHPILSYREHSFDLGMPTLVVGSGLGPCKRNPLFPPCAPQGVNHHDFFYECVAPAYHFVASDYGHLDFLDDDTKGIRGKATYCLCKNGEAREPMRKFSGGIVVAFLQAFLGDNRGALNDIMVYPSHAPVKIEPPESLVTEDVKSPEVELLRRAVCR

SEQ ID NO: 6 - protein, 313 aa, Pachira macrocarpa
MAQLLETKHDLSTVVPVFVTGKYHPTSVSVDPSNSSPSSPPKPLLIFTPSEQGTYPVILFFHGFYLRNNFYTGLLLHISSHGFIIVAPQLSNIIPPSGTEEVEHAAKVADWLPSGLPSVLPGNVEANLAKLALVGHSRGGKTAFALALGRAKTAQNFSALVGIDPVAGNRFGETSPKILTYTPGSFDLSIPVAVVGTGLGPESKGCMPCPCAPTQYNHEEFFNECKPPRVHFDAKNYGHMDTLDDNPSGFIGKLSDTICVNGEGPRDPMRRCVGGIVVAFLNYFFEAEKEDFMTIMNEPYVAPVTLDQVQFNV

SEQ ID NO: 7 - protein, 318 aa, Populus trichocarpa
MSSSSAIATVTTTVFEAGKYTTVLQKVESRTTCCTAKTSPPLPVPPPKPLLIVMPCEAGEFPLLVFLHGYLLYNSFYSQLLQHIASHGFIVIAPQLYLVAGQDSSDEIKSVAATTNWLSEGLHHLLPPHVKPNLSKLGLAGHSRGGKTAFALALEKAAATLKFSALIGVDPVDGMDKGKQTPPPVLTYVPHSFDLDMAIMVIGSGLGELKKNPLFPPCAPEGVNHKDFFKECKGPASYFVVKDYGHLDMLDDDTEGIRGKTTYCLCKNGKSREPMRKFIGGVVVAFMKAYLGGDSSDLMAIKGGQTGPVELQTVEYIL

SEQ ID NO: 8 - protein, 318 aa, Sorghum bicolor
MATTPKVLEEPPSAVITSVFQPGKLAVEVISVEHDARPTPPPIPILIAAPKDAGTYPVAILLHGFFLQNRYYEQLLKHVASFGFIMVAPQFHTSLISNSDADDIAAAAKVTDWLPEGLPTVLPTGVEADLSKLALAGHSRGGHTAFSLALGYAKTNTSSLLKFSALIGLDPVAGTGKNSQLPPAILTYEPSSFDIAVPVLVIGTGLGDERENALFPPCAPVEVNHAEFYRECRAPCYHLVTKDYGHLDMLDDDAPKLVTCLCKEGNTCKDVMRRTVAGIMVAFLKAVMGEDEDGDLKAILQHPGLAPTILDPVEYRLA

SEQ ID NO: 9 - protein, 338 aa, Sorghum bicolor
MASPVAISTTAVFKRGRHPVDTKHVDHSQVPGVPKPLMVVTPTDAGVYPVAVFLHGCSMYNSWYQTLLSHVASHGFIAVAPQLGGILPPLDMKDLKDIDATRKVTAWLADNLAHVLTNILHLHGVTPDLSRLALAGHSRGGDTAFAVALGLGSSSSSSDTTPLKFSALIGVDPVAGLSKELQLEPKVLTFEPRSLDPGMPALVVGTGLGPKGLLPCAPAGVSHGEFYDECAPPRYHVVVRDYGHLDMLDDDGVPYVISNCMCKRNTNTTKDLARRAIGGAMVAFLRAKLEDDDEDLRAVLQNSPGLSPAVLDPVEYDDDEAMDGPGCAGNNGVAGASG

SEQ ID NO: 10 - protein, 319 aa, Vitis vinifera
MALLGGNPSTQGIKLDLKTTTSVFEPGNLSVTCIRVETSNIASPPKPLLIVTPTIQGTYPVLLFLHGFELRNTFYTQLLQLISSHGYIVVAPQLYGLLPPSGIQEIKSAAAVTNWLSSGLQSVLPENVKPDLLKLALSGHSRGGKTAFALALGYADTSLNFSALLGLDPVGGLSKCSQTVPKILTYVPHSFNLAIPVCVIGTGLGDEPRNCLTCPCAPDGVNHVEFFSECKPPCSHFVTTEYGHLDMLDDHLSGCIGAISGYICKSGKGPRDPMRRCVGGLFVAFLKAYLEGQTGDFKAIVDEPDLAPVKLDPVEFIEA

SEQ ID NO: 11 - protein, 312 aa, Phyllostachys heterocycla
MAATAEIKIPSTEALEAVTSVFRPGKLAVELVPVDHNAVPTPPIPILIVAPKDAGTYPVAMLLHGFFLQNHFYEHLLKHVASHGFIMVAPQFHAICTGETEDIAAAAKVTDWLPEGLPSVLLKGVEADLSKLALAGHSRGGHTAFSLALGHGKTNLNFAALIGLDPVAGTGKSSQLPPKILTYKPSSFDVAMPVLVIGTGLGEEKKNVLFPPCAPKDVNHREFYYECKPPCYYFVTKDYGHLDMLDDDAPKFITCLCKDGDNCKDKMRRAVAGIMIAFLRAVLDEKDGDIKVILKDPGLAPVTLDPVECRLP

SEQ ID NO: 12 - protein, 319 aa, Triticum aestivum
MAAAAPAETMNKSAAGAEVPEAFTSVFQPGKLAVEAIQVDENAAPTPPIPVLIVAPKDAGTYPVAMLLHGFFLHNHFYEHLLRHVASHGFIIVAPQFSISIIPSGDAEDIAAAAKVADWLPDGLPSVLPKGVEPELSKLALAGHSRGGHTAFSLALGHAKTQLTFSALIGLDPVAGTGKSSQLQPKILTYEPSSFGMAMPVLVIGTGLGEEKKNIFFPPCAPKDVNHAEFYRECRPPCYYFVTKDYGHLDMLDDDAPKFITCVCKDGNGCKGKMRRCVAGIMVAFLNAALGEKDADLEAILRDPAVAPTTLDPVEHRVA

SEQ ID NO: 13 - protein, 318 aa, Arabidopsis thaliana
MSSSSSRNAFEDGKYKSNLLTLDSSSRCCKITPSSRASPSPPKQLLVATPVEEGDYPVVMLLHGYLLYNSFYSQLMLHVSSHGFILIAPQLYSIAGPDTMDEIKSTAEIMDWLSVGLNHFLPAQVTPNLSKFALSGHSRGGKTAFAVALKKFGYSSNLKISTLIGIDPVDGTGKGKQTPPPVLAYLPNSFDLDKTPILVIGSGLGETARNPLFPPCAPPGVNHREFFRECQGPAWHFVAKDYGHLDMLDDDTKGIRGKSSYCLCKNGEERRPMRRFVGGLVVSFLKAYLEGDDRELVKIKDGCHEDVPVEIQEFEVIM

SEQ ID NO: 14 - protein, 322 aa, Chlamydomonas reinhardtii
MPSTQFLGASTLLLFGLRAVMSSDDYIKRGDLPTSKWSGRVTLRVDSAMAVPLDVVITYPSSGAAAYPVLVMYNGFQAKAPWYRGIVDHVSSWGYTVVQYTNGGLFPIVVDRVELTYLEPLLTWLETQSADAKSPLYGRADVSRLGTMGHSRGGKLAALQFAGRTDVSGCVLFDPVDGSPMTPESADYPSATKALAAAGRSAGLVGAAITGSCNPVGQNYPKFWGALAPGSWQMVLSQAGHMQFARTGNPFLDWSLDRLCGRGTMMSSDVITYSAAFTVAWFEGIFRPAQSQMGISNFKTWANTQVAARSITFDIKPMQSPQ

SEQ ID NO: 15 - protein, 329 aa, Citrus sinensis
MAAMVDAKPAASVQGTPLLATATLPVFTRGIYSTKRITLETSSPSSPPPPKPLIIVTPAGKGTFNVILFLHGTSLSNKSYSKIFDHIASHGFIVVAPQLYTSIPPPSATNELNSAAEVAEWLPQGLQQNLPENTEANVSLVAVMGHSRGGQTAFALSLRYGFGAVIGLDPVAGTSKTTGLDPSILSFDSFDFSIPVTVIGTGLGGVARCITACAPEGANHEEFFNRCKNSSRAHFVATDYGHMDILDDNPSDVKSWALSKYFCKNGNESRDPMRRCVSGIVVAFLKDFFYGDAEDFRQILKDPSFAPIKLDSVEYIDASSMLTTTHVKV

SEQ ID NO: 16 - protein, 333 aa, Zea mays
MAASPVAIGTAVFQRGPLRVEARHVDYSQVPSVPKPLMVVAPTDAGVYPVAVFLHGCNTVNSWYESLLSHVASHGFIAVAPQLYCVTLNMNDLKDIDATRQVTAWLADKQQGLAHVLANILQLHGVRPDLSRLALAGHSRGGDTAFAVALGLGPAASDDDDNNADAGTSPAALPLKFSALIGVDPVAGLSKQAQVEPKVLTFRPRSLDPGMPALVVGTGLGPKHVGGPPCAPAGVNHAEFYDECAPPRYHVVLRDYGHMDMLDDDGVPYVINNCMCMRNTKDTKDLARRAIGGAVVAFLRATLEDDDEDLKVVLENRPGLSPAVLDPVGHDLA

SEQ ID NO: 17 - protein, 347 aa, Chenopodium album
MAKLLLLIFGVFIFVNSQAQTFPTILEKHNSEKITDVFHKGNFQVTNNPIRVKRYEFSAPEPLIIISPKEAGVYPVLLFIHGTMLSNEDYSLFFNYIASHGFIVVAPKLFRLFPPKLPSQQDEIDMAASVANWMPLYLQVVLQRYVTGVEGDLEKLAISGHSRGGKSAFALALGFSNIKLDVTFSALIGVDPVAGRSVDDRTLPHVLTYKPNSFNLSIPVTVIGSGLGNHTISCAPNHVSHQQFYDECKENSSHFVITKYGHMDMLNEFRLSPIAVTMSLMCAQSFRPKATMRRTLGGIMVAFLNAYFRDDGRQYYAIIANRSLAPTNLFAEKKGFNFGFATTYAQL

SEQ ID NO: 18 - protein, 329 aa, Picea sitchensis
MGQQGEEPWEDVFKPGRFPVRILKIPQRTTHGSTTAAAPKPLLLALPAQPGEYPVLLFFHGYLLLNSFYTQLLQHIASHGYIAIAPQMYCVTGADATPEIADAAAICNWLLQGLSSYLPDDVRPDFQNVAMAGHSRGGKVAFGLALDRTSQTTELKFSALVGVDPVDGMARGRQTQPRILTYKPHSFDSVIPTLIVGSGLGAVKRNPLFPPCAPEGVSHREFFSECSAPAYHFVASDYGHMDFLDDETGGVKGQSSYCLCKNGVAREPMRRFCGGIIVAFLNVCLQNDSGAFNDLLVHPSHAPVKLEPPESFVSEVEHQAVESLLPQTV

SEQ ID NO: 19 - DNA, 822 bp, Brassica oleracea
atggccacccccgtcgaggagggcgagtaccccgtcgtcatgctcctccacggctacctgctctacaacagcttctacagccagctcatgctccacgtcagcagctacggcttcatcgtcattgccccccagctctacaacattgccggccccgacaccatcgacgagatcaagagcaccgccgagatcatcgactggctcagcgtcggcctcaaccacttcctgcccccccaggtcacccccaacctcagcaagttcgccctcaccggccacagccgcggcggcaagaccgcctttgccgtcgccctcaagaagttcggctacagcagcgagctgaagatcagcgccatcatcggcgtcgaccccgtcgacggcaccggcaagggcaagcagacccctccccccgtcctcacctacgagcccaacagcttcaacctcgagaagatgcccgtcctcgtcatcggcagcggcctcggcgagctggcccgcaaccccctgtttcctccctgcgcccccaccggcgtcaaccaccgcgagttcttccaggagtgccagggccccgcctggcactttgtcgccaaggactacggccacctcgacatgctcgacgacgacaccaagggcctccgcggcaagagcagctactgcctctgcaagaacggcgaggagcgcaagcccatgcgccgctttatcggcggcatcgtcgtcagctttctcatggcctacctcgaggacgacgactgcgagctggtcaagatcaaggccggctgccacgagggcgtccccgtcgagatccaggagttcgaggtcaagaagtaatag

SEQ ID NO: 20 - DNA, 825 bp, Brassica oleracea
atggccggcacctaccccgtcgtcctcttcttccacggcttctacctccgcaactacttctacagcgacgtcatcaaccacgtcgccagccacggctacatcgtcgtcgccccccagctctgcaagatcctgccccctggcggccaggtcgaggtcgacgacgccggcaaggtcatcaactggaccagcaagaacctcaaggcccacctccccagcagcgtcaacgccaacggcaactacaccgccctcgtcggccacagccgcggcggcaagaccgcctttgccgtcgccctgggccacgccgccaccctcgaccccagcatcaagttcagcgccctggtcggcatcgaccctgtcgccggcatcagcaagtgcatccgcaccgaccccgagatcctcacctacaagcccgagagcttcgacctcgacatgcccgtcgccgtcatcggcaccggcctcggccccaagagcaacatgctcatgcccccctgcgcccccgccgaggtcaaccacgaggagttctacatcgagtgcaaggccaccaagggccacttcgtcgccgccgactacggccacatggacatgctcgacgacaacctccccggcttcgtcggcttcatggccggctgcatgtgcaagaacggcaagcgcaagaagtccgagatgcgcagcttcgtcggcggcattgtcgtcgcctttctcaagtacagcatctggggcgagatgagcgagatccgccagatcctcaaggaccccagcgtcagccctgcccgcctggaccctagccccgagctggaggaggcctccggctacctcgtctaatag

SEQ ID NO: 21 - DNA, 945 bp, Ricinus communis
atgagcagcagctgcgccaccgtcaccaacgtctacgagaacggcaagtacaccaccgtcgtcgccaagatcgagagcggctcctgcgcccgcagctctctccctctgcccctgccccccaagcccctcctcattgccatgcccagcgaggccggcgagttccccgtcctcatcttcctccacggctacctgctctacaacagcttctacagcctcctcatccagcacgtcgccagccacggcttcatcgtcattgccccccagctctacaccgtcgccggcgccgactccgccgacgagatcaagtgcaccgccgccatcaccaactggctcagcaagggcctccaccacgtcctgcccccccacgtccagcccaagctcagcaagctcggcctcgccggccactctcgaggcggcaaggccgcctttgccctcgccctccagaaggccggcatcagcaccgccctcaagttcagcgccctcatcggcgtcgaccccgtcgacggcatggacaagggcaagcagacccctccccctgtcctcacctacaccccccacagcttcgacctcgacatggccgccatggtcatcggcagcggcctcggcgaggtcaagcgcaaccccatgtttcccccctgcgcccccaagggcgtcaaccacgaggactttttcaaggagtgcaagaagcccgcctactacttcgtcgtcaaggactacggccacctcgacatgctcgacgacgacaccaacggcatccgcggcaaggccacgtactgcctctgcgtcaacggcaagagccgcgagcccatgcgccgctttgtcggcggcgtcctcgtcgccttcctcaaggcctacctcggcggcgactcgagcgacctcatgaccatcaccgacggccagaccggccctgtcgagctgcaggccgccgagtgctacgtctaatag

SEQ ID NO: 22 - DNA, 954 bp, Glycine max
atggcccagcgcgcccagcctgccctcgccaccaccgacgtctttcagaagggcgacatccactggaagcagttcaacgtcgagaccagcaccgccagcagcagcccccccaagcccctcctcatcttcacccccaccgtccccggcctctaccccgtcatcctgttctgccacggcttctgcatccgcaccagctactacagcaagctcctcgcccacatcgtcagccacggcttcatcctcgtcgccccccagctgttcagcatcggcgtccccatgttcggccccgaggaggtcaagtgcgagggccgcgtcgtcgactggctcgacaacggcctccagcccctcctgcccgagagcgtcgaggccaagctcgagaagctcgtcctcgtcggccacagcaagggcggcaagaccgcctttgccgtcgccctcggctactgcaagaccaagctcaagttcagcgccctcatcggcatcgaccccgtcgccggcgtcagcaagtgcaagccctgccgcagcctccccgacattctcaccggcgtcccccgcagcttcaacctcaacatccccgtcgccgtcatcggcaccggcctcggccctgagaaggccaacagcctctttcccccctgcgcccccaacggcgtcaaccacaaggagttcttctcggagtgcaagccccccagcgcctacttcgtcgccaccgactacggccacatggacatgctcgacgacgagacccccggcgtcattggcaccatgatgagcaagtgcatgtgcaagaacggcaagaagggccctcgcgacctcatgcgacgcaccgtcggcggcctcgtcgtcgcctttctgcgagcccagctcaacgagcagtggaaggacttcgacgccatcctcgccagccccaacctcgcccctgccaagctcgacgacgtccgctacctccccacctaatag

SEQ ID NO: 23 - DNA, 1032 bp, Ginkgo biloba
atggtcctcgtcaaggacgtctttagcgagggccccctgcccgtccagatcctcgccatcccccaggccaacagcagcccctgcagcaagctcgccgacaagaacggcaccgccaccacccccagcccttgccgccctcccaagcccctcctcattgccctccccagccagcacggcgactaccccctcatcctgttcttccacggctacgtcctgctcaacagcttctacagccagctcctccgccacgtcgccagccacggctacattgccattgccccccagatgtacagcgtcatcggccccaacaccacgcccgagatcgccgacgccgccgccattaccgactggctccgcgacggcctcagcgacaacctccctcaggccctgaacaaccacgtccgccccaacttcgagaagttcgtcctcgccggccacagccgcggcggcaaggtcgcctttgccctcgccctcggccgagtcagccagcccagcctcaagtacagcgccctcgtcggcctcgaccccgtcgacggcatgggcaaggaccagcagaccagccaccccatcctcagctaccgcgagcacagcttcgacctcggcatgcccaccctcgtcgtcggcagcggcctcggcccctgcaagcgcaaccccctgtttcccccctgcgcccctcagggcgtcaaccaccacgactttttctacgagtgcgtcgcccccgcctaccacttcgtcgccagcgactacggccacctcgactttctcgacgacgacaccaagggcatccgcggcaaggccacctactgcctctgcaagaacggcgaggcccgcgagcccatgcgcaagttttcgggcggcatcgtcgtcgcctttctccaggccttcctcggcgacaaccgaggcgccctcaacgacatcatggtctaccccagccacgcccccgtcaagattgagccccccgagagcctcgtcaccgaggacgtcaagagccccgaggtcgagctgctccgccgcgccgtctgccgctaatag

SEQ ID NO: 24 - DNA, 849 bp, Pachira macrocarpa
atgaacagcagcccctccagcccccccaagcccctcctcatcttcacccccagcgagcagggcacctaccccgtcatcctgttcttccacggcttctacctccgcaacaacttctacaccggcctgctcctccacatcagcagccacggcttcatcatcgtcgccccccagctcagcaacatcatcccccccagcggcaccgaggaggtcgagcacgccgccaaggtcgccgactggctccccagcggcctcccttccgtcctccccggcaacgtcgaggccaacctcgccaagctcgccctcgtcggccacagccgcggcggcaagaccgcctttgccctcgccctcggccgagccaagaccgcccagaactttagcgccctggtcggcatcgaccctgtcgccggcaaccgctttggcgagaccagccccaagatcctcacctacacccccggcagcttcgacctcagcatccccgtcgccgtcgtcggcaccggcctcggccctgagagcaagggctgcatgccctgcccctgcgcccccacccagtacaaccacgaggagttcttcaacgagtgcaagccccctcgcgtccacttcgacgccaagaactacggccacatggacaccctcgacgacaaccccagcggcttcatcggcaagctcagcgacaccatctgcgtcaacggcgagggcccccgagaccctatgcgacgctgcgtcggcggcattgtcgtcgcctttctcaactacttcttcgaggccgagaaggaggacttcatgaccatcatgaacgagccctacgtcgcccccgtcaccctcgaccaggtccagttcaacgtctaatag

SEQ ID NO: 25 - DNA, 864 bp, Populus trichocarpa
atgtgcaccgccaagaccagcccccctctccccgtcccccctcccaagcccctcctcatcgtcatgccctgcgaggccggcgagttccccctcctcgtctttctgcacggctacctgctctacaacagcttctacagccagctcctccagcacattgccagccacggcttcatcgtcattgccccccagctctacctcgtcgccggccaggacagcagcgacgagatcaagtccgtcgccgccaccaccaactggctcagcgagggcctccaccacctcctgcccccccacgtcaagcccaacctcagcaagctcggcctggccggccacagccgaggcggcaagaccgcctttgccctcgccctcgagaaggccgccgccaccctcaagttcagcgccctcattggcgtcgaccccgtcgacggcatggacaagggcaagcagacccctccccccgtcctcacctacgtcccccacagcttcgacctcgacatggccatcatggtcatcggcagcggcctcggcgagctgaagaagaaccccctgttccccccctgcgcccccgagggcgtcaaccacaaggactttttcaaggagtgcaagggccccgccagctacttcgtcgtcaaggactacggccacctcgacatgctcgacgacgacaccgagggcatccgcggcaagacgacctactgcctctgcaagaacggcaagagccgcgagcccatgcgcaagttcatcggcggcgtcgtcgtcgcctttatgaaggcctacctcggcggcgacagctccgacctcatggccattaagggcggccagaccggccccgtcgagctgcagaccgtcgagtacatcctctaatag

SEQ ID NO: 26 - DNA, 960 bp, Sorghum bicolor
atggccaccacccccaaggtcctcgaggagccccccagcgccgtcatcaccagcgtctttcagcccggcaagctcgccgtcgaggtcatcagcgtcgagcacgacgcccgccccacccctccccccattcccattctcattgccgcccctaaggacgccggcacctaccccgtcgccattctcctccacggcttttttctgcagaaccgctactacgagcagctcctcaagcacgtcgccagcttcggcttcatcatggtcgccccccagttccacaccagcctcatcagcaacagcgacgccgacgacattgccgccgccgccaaggtcaccgactggctccccgagggcctccctaccgtcctccccaccggcgtcgaggccgacctcagcaagctggccctcgccggccactctcgaggcggccacaccgcctttagcctcgccctcggctacgccaagaccaacaccagcagcctgctcaagttcagcgccctcatcggcctcgaccctgtcgccggcaccggcaagaacagccagctcccccccgccatcctcacctacgagcccagcagcttcgacattgccgtccctgtcctcgtcatcggcaccggcctcggcgacgagcgcgagaacgccctgtttcccccctgcgcccccgtcgaggtcaaccacgccgagttctaccgcgagtgccgagccccctgctaccacctcgtcaccaaggactacggccacctcgacatgctcgacgacgacgcccccaagctcgtcacgtgcctctgcaaggagggcaacacctgcaaggacgtcatgcgccgcacggtcgccggcattatggtcgcctttctcaaggccgtcatgggcgaggacgaggacggcgacctcaaggccatcctccagcaccccggcctcgcccccaccatcctggaccccgtcgagtaccgcctcgcctaatag

SEQ ID NO: 27 - DNA, 1020 bp, Sorghum bicolor
atggccagccccgtcgccatcagcaccaccgccgtctttaagcgaggccgccaccccgtcgacaccaagcacgtcgaccacagccaggtccccggcgtccccaagcctctcatggtcgtcacccccaccgatgccggcgtctaccccgtggctgtctttctccacggctgctccatgtacaacagctggtatcagaccctcctcagccacgtcgcctcccacggctttattgccgtcgctccccagctcggcggcattctgccccccctggacatgaaggacctcaaggacatcgacgccacccgcaaggtcaccgcctggctcgccgataacctcgcccacgtcctcaccaacatcctccacctccacggcgtcacgcccgacctgtctcgactcgctctcgctggccactctcgaggcggcgacactgcctttgctgtcgctctcggcctcggcagcagcagctctagcagcgacaccacccccctcaagttcagcgccctcatcggcgtcgaccctgtcgccggcctcagcaaggagcttcagctcgagcccaaggtcctcaccttcgagccccgaagcctcgaccctggcatgcctgctctcgtcgtcggcactggcctcggccctaagggcctccttccttgcgctcctgctggcgtcagccacggcgagttctacgacgagtgcgcccctccccgctaccacgtcgtcgtccgagactacggccacctcgacatgctcgacgacgatggcgtcccctacgtcatcagcaactgcatgtgcaagcgcaacaccaacaccaccaaggacctcgcccgacgcgccattggcggcgctatggtcgcctttctgcgcgccaagctcgaggatgacgacgaggacctccgcgccgtcctccagaacagccctggcctctctcctgccgtcctggacccggtcgagtacgacgatgacgaggccatggacggccctggctgcgctggcaacaacggcgttgccggcgctagcggctaatag

SEQ ID NO: 28 - DNA, 855 bp, Vitis vinifera
atgaccagcaacattgccagcccccccaagcccctcctcatcgtcacccccaccatccagggcacctaccccgtcctgctgttcctccacggcttcgagctgcgcaacaccttctacacccagctcctccagctcatcagcagccacggctacatcgtcgtcgccccccagctctacggcctcctgccccccagcggcatccaggagattaagagcgccgccgccgtcaccaactggctcagcagcggcctccagagcgtcctccccgagaacgtcaagcccgacctcctcaagctcgccctcagcggccacagccgcggcggcaagaccgcctttgccctcgccctcggctacgccgacaccagcctcaacttcagcgccctcctcggcctcgaccccgtcggcggcctcagcaagtgcagccagaccgtccccaagatcctcacctacgtcccccacagcttcaacctcgccatccccgtctgcgtcatcggcaccggcctcggcgacgagccccgcaactgcctgacctgcccttgcgcccccgacggcgtcaaccacgtcgagttcttcagcgagtgcaagcccccctgcagccacttcgtcaccaccgagtacggccacctcgacatgctcgacgaccacctcagcggctgcatcggcgccattagcggctacatctgcaagagcggcaagggccctcgcgaccctatgcgacgctgcgtcggcggcctgtttgtcgccttcctcaaggcctacctcgagggccagaccggcgacttcaaggccattgtcgacgagcctgacctcgcccccgtcaagctggaccccgtcgagttcatcgaggcctaatag

SEQ ID NO: 29 - DNA, 942 bp, Phyllostachys sp.
atggccgccaccgccgagatcaagatccccagcaccgaggctctcgaggccgtcaccagcgtctttcgccccggcaagctggccgtcgagctggtccccgtcgaccacaacgccgtccccaccccccccatccccatcctcatcgtcgcccctaaggacgccggcacctaccccgtcgctatgctcctccacggcttttttctgcagaaccacttctacgagcacctcctcaagcacgtcgccagccacggcttcatcatggtcgccccccagttccacgccatctgcaccggcgagaccgaggacattgctgctgccgccaaggtcaccgactggctccccgagggcctccccagcgtcctcctgaagggcgtcgaggctgacctcagcaagctcgccctcgccggccactctcgcggcggccacactgccttcagcctcgccctcggccacggcaagaccaacctcaacttcgccgccctcatcggcctcgaccctgtcgctggcaccggcaagagcagccagctgccccccaagatcctcacctacaagcccagcagcttcgacgtcgccatgcctgtcctcgtcatcggcacgggcctcggcgaggagaagaagaacgtcctcttcccgccctgcgcccccaaggacgtcaaccaccgcgagttctactacgagtgcaagcccccctgctactacttcgtcaccaaggactacggccacctcgacatgctcgacgacgacgcccccaagttcatcacgtgcctctgcaaggacggcgacaactgcaaggacaagatgcgccgcgccgtcgccggcatcatgatcgcctttctccgcgccgtcctcgacgagaaggatggcgacatcaaggtcatcctcaaggaccccggcctggcccccgtcactctggaccccgtcgagtgccgcctcccctaatag

SEQ ID NO: 30 - DNA, 822 bp, Arabidopsis sp.
atggccacccccgtcgaggagggcgactaccccgtcgtcatgctcctccacggctacctgctctacaacagcttctacagccagctcatgctccacgtcagcagccacggcttcatcctgatcgccccccagctctacagcattgccggccccgacaccatggacgagatcaagagcaccgccgagatcatggactggctcagcgtcggcctcaaccacttcctgcccgcccaggtcacccccaacctcagcaagttcgccctcagcggccacagccgaggcggcaagaccgccttcgccgtcgccctcaagaagttcggctacagcagcaacctcaagatcagcaccctcatcggcatcgaccccgtcgacggcaccggcaagggcaagcagacccccccccccgtcctcgcctacctccccaacagcttcgacctcgacaagacccccatcctcgtcatcggcagcggcctcggcgagactgcccgcaaccctctgttccctccctgcgccccccctggcgtcaaccaccgcgagttcttccgcgagtgccagggccccgcctggcacttcgtcgccaaggactacggccacctcgacatgctcgacgacgacaccaagggcatccgcggcaagagcagctactgcctctgcaagaacggcgaggagcgccgccctatgcgccgctttgtcggcggcctcgtcgtcagcttcctcaaggcctacctcgagggcgacgaccgcgagctggtcaagatcaaggacggctgccacgaggacgtccccgtcgagatccaggagttcgaggtcatcatgtaatag

SEQ ID NO: 31 - DNA, 933 bp, Citrus sinensis
atggccaccctccccgtctttacccgcggcatctacagcaccaagcgcatcaccctcgagaccagctcccccagctcgccccctccgcccaagcccctcatcatcgtcacccccgccggcaagggcaccttcaacgtcatcctgttcctccacggcaccagcctcagcaacaagagctacagcaagatcttcgaccacattgccagccacggcttcatcgtcgtcgccccccagctctacaccagcattccccccccctcggccaccaacgagctgaacagcgccgccgaggtcgccgagtggctccctcagggcctccagcagaacctccccgagaacaccgaggccaacgtcagcctcgtcgccgtcatgggccacagccgaggcggccagaccgccttcgccctcagcctccgctacggcttcggcgccgtcatcggcctcgaccccgtcgccggcaccagcaagaccaccggcctggaccccagcatcctgtccttcgacagcttcgacttcagcatccctgtcaccgtcatcggcaccggcctcggcggcgtcgcccgatgcattaccgcctgcgcccctgagggcgccaaccacgaggagttcttcaaccgctgcaagaacagcagccgcgcccacttcgtcgccaccgactacggccacatggacatcctcgacgacaaccccagcgacgtcaagagctgggccctcagcaagtacttctgcaagaacggcaacgagagccgcgaccccatgcgccgctgcgtctccggcattgtcgtcgcctttctcaaggacttcttctacggcgacgccgaggacttccgccagatcctcaaggacccctcgttcgcccccatcaagctcgactcggtcgagtacatcgacgccagcagcatgctcaccaccacccacgtcaaggtctaatag

SEQ ID NO: 32 - DNA, 1005 bp, Zea mays
atggccgccagccccgtcgccatcggcaccgccgtctttcagcgcggccccctccgcgtcgaggcccgccacgtcgactacagccaggtccccagcgtccccaagcccctcatggtcgtcgcccccaccgacgccggcgtctaccccgtcgccgtctttctccacggctgcaacaccgtcaacagctggtacgagagcctcctcagccacgtcgccagccacggctttatcgccgtcgccccccagctctactgcgtcaccctcaacatgaacgacctcaaggacatcgacgccacccgccaggtcaccgcctggctcgccgacaagcagcagggcctcgcccacgtcctcgccaacatcctccagctccacggcgtccgccccgacctcagccgcctcgccctcgccggccacagccgcggcggcgacaccgcctttgccgtcgccctcggcctcggccccgccgccagcgacgacgacgacaacaacgccgacgccggcaccagccccgccgccctgcccctcaagttcagcgccctcatcggcgtcgaccccgtcgccggcctcagcaagcaggcccaggtcgagcccaaggtcctcacctttcgcccccgcagcctcgaccccggcatgcccgccctcgtcgtcggcaccggcctgggccccaagcacgtcggcggccccccctgcgcccccgccggcgtcaaccacgccgagttctacgacgagtgcgcccccccccgctaccacgtcgtcctccgcgactacggccacatggacatgctcgacgacgacggcgtcccctacgtcatcaacaactgcatgtgcatgcgcaacaccaaggacacgaaggacctcgcccgccgcgccattggcggcgccgtcgtcgccttcctccgcgccaccctcgaggacgacgacgaggacctcaaggtcgtcctcgagaaccgccccggcctctcccccgccgtcctggaccccgtcggccacgacctcgcctaatag

SEQ ID NO: 33 - DNA, 981 bp, Triticum aestivum
atggtcgccgtcgagaagcgcgccgctgccgcccctgccgagaccatgaacaagtccgccgccggcgccgaggtccctgaggccttcaccagcgtctttcagcccggcaagctcgccgtcgaggccatccaggtcgacgagaacgccgcccccacccctcccatccccgtcctcatcgtcgcccccaaggacgccggcacctaccccgtcgccatgctcctccacggcttttttctccacaaccacttctacgagcacctcctccgccacgtcgccagccacggcttcatcattgtcgccccccagttcagcatcagcatcatccccagcggcgacgccgaggacattgccgccgccgccaaggtcgctgactggctccccgacggcctgccctccgtcctccccaagggcgtcgagcccgagctgtccaagctcgccctcgccggccactctcgcggaggccacaccgcctttagcctcgccctcggccacgccaagacccagctcaccttcagcgccctcatcggcctcgaccctgtcgccggcaccggcaagagcagccagctccagcccaagatcctcacctacgagcccagcagctttggcatggccatgccggtcctcgtcatcggcaccggcctcggcgaggagaagaagaacatcttcttcccgccctgcgcccctaaggacgtcaaccacgccgagttctaccgcgagtgccgccccccctgctactacttcgtcaccaaggactacggccacctcgacatgctcgacgacgacgcccccaagttcatcacgtgcgtctgcaaggacggcaacggctgcaagggcaagatgcgccgctgcgtcgccggcatcatggtcgcctttctcaacgccgccctgggcgagaaggacgccgacctcgaggccattctccgcgaccccgccgtcgcccctactaccctggaccccgtcgagcaccgcgtcgcctaatag

SEQ ID NO: 34 - DNA, 837 bp, Chlamydomonas reinhardtii
atgagcgccatggccgtccccctcgacgtcgtcatcacctaccccagctctggcgccgccgcctaccccgtcctcgtcatgtacaacggcttccaggccaaggccccctggtaccgcggcatcgtcgaccacgtcagcagctggggctacaccgtcgtccagtacaccaacggcggcctcttccccatcgtcgtcgaccgcgtcgagctgacctacctcgagcccctcctgacctggctcgagactcagagcgccgacgccaagagccccctctacggccgagccgacgtcagccgcctcggcaccatgggccacagccgaggcggcaagctcgccgccctccagtttgccggccgaacggacgtctccggctgcgtcctcttcgaccccgtcgacggcagccccatgacccccgagagcgccgactaccccagcgccactaaggccctcgccgctgctggccgatctgctggcctcgtcggcgccgccatcaccggcagctgcaaccccgtcggccagaactaccccaagttctggggcgccctcgcccctggcagctggcagatggtcctcagccaggccggccacatgcagttcgcccgcaccggcaacccgttcctcgactggtccctcgaccgcctctgcggccgaggcaccatgatgtccagcgacgtcatcacgtacagcgccgccttcaccgtcgcctggttcgagggcatcttccgccctgcccagagccagatgggcatcagcaacttcaagacctgggccaacacccaggtcgccgcccgatccatcaccttcgacatcaagcccatgcagagcccccagtaatag

SEQ ID NO: 35 - DNA, 993 bp, Picea sitchensis
atgggccagcagggcgaggagccctgggaggacgtctttaagcctggccgcttccccgtccgcattctcaagatcccccagcgcaccacccacggcagcaccaccgctgctgctcccaagcctctcctcctcgccctgcctgcccagcccggcgagtaccccgtcctcctgttcttccacggctacctgctcctcaacagcttctacacccagctcctccagcacattgccagccacggctacattgccattgccccccagatgtactgcgtcactggcgccgacgctacgcctgagattgccgacgctgccgctatctgcaactggctccttcagggcctcagcagctacctccccgacgacgtccgccccgacttccagaacgtcgccatggctggccactcccgaggcggcaaggtggccttcggcctggccctcgaccgaaccagccagaccaccgagctgaagttcagcgccctcgtcggcgtggaccctgtcgatggcatggcccgaggccgacagacccagccccgcatcctcacctacaagccccacagcttcgacagcgtcatccccaccctcatcgtcggctcgggcctcggcgccgtcaagcgcaaccccctgttcccgccctgcgctcccgagggcgtcagccaccgcgagttcttcagcgagtgcagcgctcccgcctaccacttcgtcgccagcgactacggccacatggactttctcgacgacgagactggcggcgtcaagggccagtcctcctactgcctctgcaagaacggcgtcgcccgcgagcccatgcgccgcttttgcggcggcatcatcgtcgcctttctcaacgtctgcctccagaacgacagcggcgccttcaacgacctcctcgtccaccccagccacgcccccgtgaagctcgagccccccgagagcttcgtcagcgaggtcgagcaccaggccgtcgagagcctcctgccccagaccgtctaatag

SEQ ID NO: 36 - protein, 6 aa, Artificial Sequence
  Amino acid sequence signature motif
GHSXGG

SEQ ID NO: 37 - protein, 5 aa, Artificial Sequence
  Amino acid sequence signature motif
DPVXG

SEQ ID NO: 38 - protein, 5 aa, Artificial Sequence
  Amino acid sequence signature motif
YGHXD

SEQ ID NO: 39 - DNA, 85 bp, Artificial Sequence
  Signal peptide-encoding sequence
caccatgattgtcggcattctcaccacgctggctacgctggccacactcgcagctagtgtgcctctagaggagcggactagtgcg

SEQ ID NO: 40 - DNA, 67 bp, Artificial Sequence
  Signal peptide-encoding sequence
caccatgtatcggaagttggccgtcatctcggccttcttggccacagctcgtgctcagtcgactagt

SEQ ID NO: 41 - DNA, 152 bp, Artificial Sequence
  Signal peptide-encoding sequence
caccatgttctctggacggtttggagtgcttttgacagcgcttgctgcgctgggtgctgccgcgccggcaccgcttgctgtgcggagtaggtgtgcccgatgtgagatggttggatagcactgatgaagggtgaataggtgtctcgactagt

SEQ ID NO: 42 - DNA, 76 bp, Artificial Sequence
  Signal peptide-encoding sequence
caccatgcacgtcctgtcgactgcggtgctgctcggctccgttgccgttcaaaaggtcctgggaagaccaactagt

SEQ ID NO: 43 - DNA, 377 bp, Artificial Sequence
  Signal peptide-encoding sequence
caccatgattgtcggcattctcaccacgctggctacgctggccacactcgcagctagtgtgcctctagaggagcggcaagcttgctcaagcgtctggtaattatgtgaaccctctcaagagacccaaatactgagatatgtcaaggggccaatgtggtggccagaattggtcgggtccgacttgctgtgcttccggaagcacatgcgtctactccaacgactattactcccagtgtcttcccggcgctgcaagctcaagctcgtccacgcgcgccgcgtcgacgacttctcgagtatcccccacaacatcccggtcgagctccgcgacgcctccacctggttctactactaccagagtacctccagtcggaactagt

SEQ ID NO: 44 - DNA, 286 bp, Artificial Sequence
  Signal peptide-encoding sequence
caccatgaacaagtccgtggctccattgctgcttgcagcgtccatactatatggcggcgccgctgcacagcagactgtctggggccagtgtggaggtattggttggagcggacctacgaattgtgctcctggctcagcttgttcgaccctcaatccttattatgcgcaatgtattccgggagccactactatcaccacttcgacccggccaccatccggtccaaccaccaccaccagggctacctcaacaagctcatcaactccacccacgagctctgggactagt

SEQ ID NO: 45 - DNA, 76 bp, Artificial Sequence
  Signal peptide-encoding sequence
caccatggcgccctcagttacactgccgttgaccacggccatcctggccattgcccggctcgtcgccgccactagt

SEQ ID NO: 46 - DNA, 73 bp, Artificial Sequence
  Signal peptide-encoding sequence
caccatgaaggtctctcgagtccttgcccttgtcctgggggccgtcatccctgcccatgctgcctttactagt

SEQ ID NO: 47 - DNA, 73 bp, Artificial Sequence
  Signal peptide-encoding sequence
caccatggttgccttttccagcctcatctgcgctctcaccagcatcgccagtactctggcgatgcccactagt

SEQ ID NO: 48 - DNA, 64 bp, Artificial Sequence
  Signal peptide-encoding sequence
caccatgaaagcaaacgtcatcttgtgcctcctggcccccctggtcgccgctctccccactagt

SEQ ID NO: 49 - protein, 122 aa, Arabidopsis thaliana
FALSGHSRGGKTAFAVALKKFGYSSNLKISTLIGIDPVDGTGKGKQTPPPVLAYLPNSFDLDKTPILVIGSGLGETARNPLFPPCAPPGVNHREFFRECQGPAWHFVAKDYGHLDMLDDDTK

SEQ ID NO: 50 - protein, 122 aa, Brassica oleracea
FALTGHSRGGKTAFAVALKKFGYSSELKISAIIGVDPVDGTGKGKQTPPPVLTYEPNSFNLEKMPVLVIGSGLGELARNPLFPPCAPTGVNHREFFQECQGPAWHFVAKDYGHLDMLDDDTK

SEQ ID NO: 51 - protein, 121 aa, Ricinus communis
LGLAGHSRGGKAAFALALQKAGISTALKFSALIGVDPVDGMDKGKQTPPPVLTYTPHSFDLDMAAMVIGSGLGEVKRNPMFPPCAPKGVNHEDFFKECKKPAYYFVVKDYGHLDMLDDDTN

SEQ ID NO: 52 - protein, 119 aa, Populus trichocarpa
LGLAGHSRGGKTAFALALEKAAATLKFSALIGVDPVDGMDKGKQTPPPVLTYVPHSFDLDMAIMVIGSGLGELKKNPLFPPCAPEGVNHKDFFKECKGPASYFVVKDYGHLDMLDDDTE

SEQ ID NO: 53 - protein, 120 aa, Ginkgo biloba
FVLAGHSRGGKVAFALALGRVSQPSLKYSALVGLDPVDGMGKDQQTSHPILSYREHSFDLGMPTLVVGSGLGPCKRNPLFPPCAPQGVNHHDFFYECVAPAYHFVASDYGHLDFLDDDTK

SEQ ID NO: 54 - protein, 119 aa, Phyllostachys heterocycla
LALAGHSRGGHTAFSLALGHGKTNLNFAALIGLDPVAGTGKSSQLPPKILTYKPSSFDVAMPVLVIGTGLGEEKKNVLFPPCAPKDVNHREFYYECKPPCYYFVTKDYGHLDMLDDDAP

SEQ ID NO: 55 - protein, 119 aa, Triticum aestivum
LALAGHSRGGHTAFSLALGHAKTQLTFSALIGLDPVAGTGKSSQLQPKILTYEPSSFGMAMPVLVIGTGLGEEKKNIFFPPCAPKDVNHAEFYRECRPPCYYFVTKDYGHLDMLDDDAP

SEQ ID NO: 56 - protein, 123 aa, Sorghum bicolor
LALAGHSRGGHTAFSLALGYAKTNTSSLLKFSALIGLDPVAGTGKNSQLPPAILTYEPSSFDIAVPVLVIGTGLGDERENALFPPCAPVEVNHAEFYRECRAPCYHLVTKDYGHLDMLDDDAP

SEQ ID NO: 57 - protein, 122 aa, Sorghum bicolor
LALAGHSRGGDTAFAVALGLGSSSSSSDTTPLKFSALIGVDPVAGLSKELQLEPKVLTFEPRSLDPGMPALVVGTGLGPKGLLPCAPAGVSHGEFYDECAPPRYHVVVRDYGHLDMLDDDGV

SEQ ID NO: 58 - protein, 135 aa, Zea mays
LALAGHSRGGDTAFAVALGLGPAASDDDDNNADAGTSPAALPLKFSALIGVDPVAGLSKQAQVEPKVLTFRPRSLDPGMPALVVGTGLGPKHVGGPPCAPAGVNHAEFYDECAPPRYHVVLRDYGHMDMLDDDGV

SEQ ID NO: 59 - protein, 121 aa, Brassica oleracea
TALVGHSRGGKTAFAVALGHAATLDPSIKFSALVGIDPVAGISKCIRTDPEILTYKPESFDLDMPVAVIGTGLGPKSNMLMPPCAPAEVNHEEFYIECKATKGHFVAADYGHMDMLDDNLP

SEQ ID NO: 60 - protein, 111 aa, Citrus sinensis
VAVMGHSRGGQTAFALSLRYGFGAVIGLDPVAGTSKTTGLDPSILSFDSFDFSIPVTVIGTGLGGVARCITACAPEGANHEEFFNRCKNSSRAHFVATDYGHMDILDDNPS

SEQ ID NO: 61 - protein, 118 aa, Pachira macrocarpa
LALVGHSRGGKTAFALALGRAKTAQNFSALVGIDPVAGNRFGETSPKILTYTPGSFDLSIPVAVVGTGLGPESKGCMPCPCAPTQYNHEEFFNECKPPRVHFDAKNYGHMDTLDDNPS

SEQ ID NO: 62 - protein, 119 aa, Vitis vinifera
LALSGHSRGGKTAFALALGYADTSLNFSALLGLDPVGGLSKCSQTVPKILTYVPHSFNLAIPVCVIGTGLGDEPRNCLTCPCAPDGVNHVEFFSECKPPCSHFVTTEYGHLDMLDDHLS

SEQ ID NO: 63 - protein, 121 aa, Glycine max
LVLVGHSKGGKTAFAVALGYCKTKLKFSALIGIDPVAGVSKCKPCRSLPDILTGVPRSFNLNIPVAVIGTGLGPEKANSLFPPCAPNGVNHKEFFSECKPPSAYFVATDYGHMDMLDDETP

SEQ ID NO: 64 - protein, 116 aa, Chenopodium album
LAISGHSRGGKSAFALALGFSNIKLDVTFSALIGVDPVAGRSVDDRTLPHVLTYKPNSFNLSIPVTVIGSGLGNHTISCAPNHVSHQQFYDECKENSSHFVITKYGHMDMLNEFRL

SEQ ID NO: 65 - protein, 19 aa, Artificial Sequence
  Consensus sequence
LALAGHSRGGKTAFALALG

SEQ ID NO: 66 - protein, 15 aa, Artificial Sequence
  Consensus sequence
LKFSALIGLDPVAGL

SEQ ID NO: 67 - protein, 4 aa, Artificial Sequence
  Consensus sequence
ILTY

SEQ ID NO: 68 - protein, 4 aa, Artificial Sequence
  Consensus sequence
SFDL

SEQ ID NO: 69 - protein, 12 aa, Artificial Sequence
  Consensus sequence
MPVLVIGTGLGE

SEQ ID NO: 70 - protein, 7 aa, Artificial Sequence
  Consensus sequence
LFPPCAP

SEQ ID NO: 71 - protein, 4 aa, Artificial Sequence
  Consensus sequence
GVNH

SEQ ID NO: 72 - protein, 4 aa, Artificial Sequence
  Consensus sequence
YHFV

SEQ ID NO: 73 - protein, 12 aa, Artificial Sequence
  Consensus sequence
KDYGHLDMLDDD